(19) United States
(12) Patent Application Publication
(10) Pub. No.: US 2011/0182652 A1
(43) Pub. Date: Jul. 28, 2011

(54) Wearable Sanitizing Gel Dispenser, Kit, and Associated Methods

(76) Inventors: Hannah Chung, Evanston, IL (US); Katherine Porter, Evanston, IL (US); Mert Iseri, Evanston, IL (US); Yuri Malina, Evanston, IL (US); Ghislaine Maxwell, Miami Beach, FL (US)

(21) Appl. No.: 12/945,343

(22) Filed: Nov. 12, 2010

Related U.S. Application Data
(60) Provisional application No. 61/297,346, filed on Jan. 22, 2010.

(51) Int. Cl. B67D 7/84

(52) U.S. Cl. 401/218; 401/220

(57) ABSTRACT

Embodiments of the present invention relate to wearable sanitizing gel dispensers. An embodiment of a dispenser includes an outer gel container adapted to house gel therein, or alternatively an inner gel container to house gel and outer gel container housing adapted to house the inner gel container. Connected to the outer gel housing is a dispenser body housing an ellipsoidal roller adapted to dispense gel to hands of healthcare personnel when actuated by a selective hand motion. The dispenser can also include a flexible fabric housing positioned to house the inner gel container and adapted so that the outer surfaces of the flexible fabric housing are made out of an antibacterial fabric to receive gel residue from hand of healthcare personnel when applied thereto. Embodiments of methods of actuating and using a wearable sanitizing gel dispenser and embodiments of kits for wearable sanitizing gel also are included.

### CROSS-REFERENCE TO RELATED APPLICATION

This application claims benefit of, priority to, and is related to U.S. Provisional Patent Application No. 61/297346 filed on Jan. 22, 2010 titled, “Wearable Gel Dispenser,” which is incorporated herein by reference in its entirety.

### FIELD OF INVENTION

Embodiments of the present invention relate generally to dispensers. More particularly, embodiments of the present invention relate to sanitizing gel dispensers.

### BACKGROUND

One of the main causes of infectious diseases, as well as other types of illnesses, in humans results from disease-carrying organisms on hands transferring to other body parts such as the mouth, eyes, and nose. The spread of disease-causing organisms is especially problematic in hospitals or other healthcare environments. Accordingly, sanitizing gel has become an important staple to hand hygiene, especially in the healthcare environment.

Sanitizing gel allows users to disinfect their hands without the use of running water. Many sanitizing dispensers, however, are inconvenient because they are held or rest on a surface to dispense the sanitizer. For example, pump gel dispensers often include a horizontal spout having a tip of the spout directed downward. To actuate such dispensers, the user might pump the spout with one hand, and extend the other hand underneath the spout to receive the sanitizer. When using such dispensers, the force imparted on the pump head can cause the dispenser to tip over if it is not braced or steadied. As a result many users steady the dispenser with one hand while pumping it with the other. These dispensers, however, often are inconvenient, especially for professionals who must clean their hands frequently and move from room to room.

Current wearable gel dispensers often require the user to actuate a pump or squeeze the bladder of a container to dispense the gel. Examples of such dispensers are described in U.S. Patent Application 2006/0289567 by Shoham et al. titled “Wearable Disinfecting Gel Dispenser” and U.S. Pat. No. 6,983,864 by Ronald Cagle, titled “Fluid Dispenser Assembly.” Because such dispensers require the user to use an unnatural motion to dispense the gel, these dispensers may be time-consuming to use and burdensome to handle, especially for personnel that have a need or desire to sanitize their hands frequently.

In order to achieve the frequency of hand sanitizing that the healthcare environment demands, hand sanitizing should be intuitive and natural. Accordingly, there is still a need for a cost-effective and time-saving wearable sanitizing gel dispenser that is appropriate for the healthcare environment.

### SUMMARY OF THE INVENTION

In view of the foregoing, embodiments of the present invention advantageously dispense sanitizing gel to one or more hands of a healthcare personnel using a wearable sanitizing gel dispenser actuated by perhaps a more intuitive and natural hand motion. Embodiments also allow amounts of excess gel residue to be removed readily from hands of healthcare personnel during this perhaps more intuitive and natural hand motion to thereby reduce the desire or need to wipe the residue on clothing, separate cloths or other wipes, or other items, including another hand, for example. Although referred to by specific example, various other personnel, e.g., industry, office, manufacturing, government, can also use the dispensers as will be understood by those skilled in the art, and the healthcare personnel example and reference herein includes these other areas of use and other personnel in these environments.

An embodiment of the present invention includes a sanitizing gel dispenser to dispense sanitizing gel to the hands of healthcare personnel when the dispenser is attached to clothing or clothing accessories of healthcare personnel. Healthcare organizations highly recommend, or even require, that healthcare personnel sanitize their hands before and after every contact with a patient. Embodiments of the present invention can worn by healthcare personnel throughout their working shift to sanitize their hands and assist in meeting hand hygiene guidelines.

Embodiments of the present invention includes, for example, an outer gel container housing adapted to store sanitizing gel therein. The outer gel container housing can be disposable, replaceable or refillable and can include, for example, an aperture to allow the gel to exit from the outer gel container housing. A dispenser body, according to embodiments of the present invention, is connected to the outer gel container housing and receives gel from the aperture therein.

In an embodiment of a dispenser, the aperture initially can be covered, for example, by a thin piece of penetrable material to secure the gel within the container. This penetrable material then can be pierced by a valve disposed on an end portion of the dispenser body when the outer gel container housing is positioned to connect to or be mounted on the dispenser body. The valve, according to embodiments of the invention, matingly connects to the aperture so that the dispensing body is adapted to receive gel flowing from the outer gel container housing. The dispenser body can include, for example, a linking mechanism to allow the dispenser body to selectively detach from the outer gel container housing. The dispenser body can also include a roller seat formed in a front face of the dispenser body to secure an ellipsoidal roller within the dispenser body.

According to embodiments of the invention, inner surfaces of the valve extend downwardly within the inner surfaces of the dispenser body when the outer gel container housing is mounted to the dispenser body. The inner surfaces of the valve, for example, can connect to a throat positioned within the dispenser body through which the gel fluidly travels to the roller seat and therefrom to an ellipsoidal roller. The ellipsoidal roller is connected to and seated in the roller seat and is shaped to allow gel to be readily dispensed to one or more hands of healthcare personnel by a selective hand motion by healthcare personnel. The selective hand motion, for example, can actuate the ellipsoidal roller to rollingly supply gel to the ellipsoidal roller and therefrom to one or more hands of healthcare personnel. The dispenser also can include clothing attachment member, as will be understood by those skilled in the art, (e.g., a clip, hook and loop fastener or Lanyard) to allow the dispenser to attached to clothing or clothing accessories of healthcare personnel in an orientation so as to allow gravity to cause the gel to flow toward the aperture.

Embodiments of the present invention can also include a release mechanism connected to the dispenser body to selectively detach the detachable outer housing from the linking mechanism of the dispenser body. Accordingly, embodiments of the present invention can be conveniently detached to replace one or more parts of the dispenser or refill the outer gel container housing.

In alternative to the gel being stored in the outer gel container housing, embodiments of the present invention include an inner gel container adapted to store the gel therein and having an aperture through which the gel can exit through. Additionally, an outer gel container housing can be adapted to position and house the inner gel container. In this embodiment, the valve matingly connects with the aperture of inner gel container to allow fluid communication so that the dispenser body is adapted to receive gel from the inner gel container and is dispensed by the dispenser body through the ellipsoidal roller.

Embodiments of the present invention can also include, for example, a flexible fabric housing positioned to house the inner gel container or to define the outer gel housing container in other embodiments where an inner gel container is not used or needed. This flexible fabric housing can replace or be another embodiment of the outer gel container housing as needed. In addition, either the ellipsoidal roller can be used in this embodiment or dispensing sponge, e.g., elongated. In this embodiment, either the ellipsoidal roller or the dispensing sponge can be connected to and positioned to extend along an end portion of the flexible fabric housing so that outer surfaces of the flexible fabric housing is adapted so that the outer surfaces of the flexible fabric housing receive gel residue from one or more hands of healthcare personnel when using the wearable gel dispenser. According to an embodiment of the invention, at least the outer surfaces of the flexible fabric housing include an antibacterial fabric as will be understood by those skilled in the art. The antibacterial fabric also can sanitize the hands of healthcare personnel as a primary or secondary sanitizer.

According to an embodiment of the present invention, the dispensing sponge preferably is in fluid communication with the aperture of the inner gel container and adapted to saturate the sponge with the gel to dispense the gel to hands of healthcare personnel. The size of the wearable dispensers, according to embodiments of the present invention, is preferably such that it readily can fit within the average size hand

Embodiments of the present invention also include a dispenser kit having a plurality of assembled disposable wearable gel dispensers positioned within a container. The plurality of disposable wearable gel dispensers are adapted to dispense sanitizing gel to one or more hands of healthcare personnel when attached to clothing or clothing accessories of healthcare personnel. Each of the plurality of disposable wearable gel dispensers can be disposed, as will be understood by those skilled in the art, after a predetermined amount of individual dispenses, e.g., fifty individual dispenses, or after a predetermined amount of time such as after a shift of a healthcare personnel. The kit, for example, can include one dispenser body and plurality of disposable, replaceable, or refillable gel housing containers, e.g., pouches, housings, or casings, that are then used to replace a used or empty container when healthcare personnel or other users use the sanitizing gel there.

Another embodiment of the present invention includes a dispenser kit of a wearable gel dispenser having a plurality of replaceable and refillable outer gel container housing and a dispenser body positioned within a container. Each of the plurality of outer gel container housing are adapted to position and house gel therein. The dispenser body, according to embodiments of the invention, is adapted to connect to the plurality of outer gel container housing to dispense sanitizing gel to one or more hands of healthcare personnel when attached to clothing or clothing accessories of healthcare personnel.

Embodiments of the present invention also include methods of dispensing sanitizing gel with a sanitizing gel dispenser to hands of an individual, such as a health personnel. Embodiments of a method include the step of actuating an ellipsoidal roller using a selective hand motion by healthcare personnel to supply gel to hands of healthcare personnel. In the alternative, embodiments of a method can include the step of squeezing a dispensing sponge to supply gel to hands of healthcare personnel. Another embodiment of a method includes wiping one or more hands on the outer surfaces of the sponge to sanitize said hands. Yet another embodiment of the method can include the step of apply one or more hands of a healthcare personnel to the outer surfaces of a flexible fabric housing to sanitize said hands.

An embodiment of a method also can include the step of attaching a dispenser according to any of the dispenser embodiments discussed herein to the clothing or clothing accessories of healthcare personnel. Embodiments of the methods, for example, can include the step of providing a selective portion of gel to the hands of healthcare personnel—the selective portion of the gel being significantly less than the amount of gel capable of being stored in the dispenser.

### BRIEF DESCRIPTION OF THE DRAWINGS

So that the manner in which the features and advantages of the invention, as well as others which will become apparent are attained and can be understood in more detail, a more particular description of the invention briefly summarized above may be had by reference to embodiments thereof which are illustrated in the appended drawings, which drawings form a part of this specification. It is to be noted, however, that the drawings illustrate only embodiments of the present invention and, therefore, are not to be considered limiting of its scope as the invention may extend to other equally effective embodiments.

FIG. 1 is an environmental view of healthcare personnel wearing a dispenser according to an embodiment of the present invention;

FIG. 2 is an environmental view of healthcare personnel using a dispenser according to an embodiment of the present invention;

FIG. 2A is an enlarged elevational view of FIG. 2 according to an embodiment of the present invention;

FIG. 3 is an environmental view of healthcare personnel sanitizing hands according to an embodiment of the present invention;

FIG. 4 is a exploded perspective view of a dispenser according to an embodiment of the present invention;

FIG. 5 is an perspective view of the dispenser according to an embodiment of the present invention;

FIG. 6 is an enlarged view of a portion of an outer gel container housing having a valve being connected to a dispenser body according to an embodiment of the present invention;

FIG. 7 is an sectional view of a dispenser taken along line 7-7 of FIG. 5 according to an embodiment of the present invention;

FIG. 8 is an exploded perspective view of a dispenser according to another embodiment of the present invention;

FIG. 9 is perspective view of a dispenser without an ellipsoidal roller positioned therein a roller seat of a dispenser body according to another embodiment of the present invention;

FIG. 10 is perspective view of a dispenser without an elliptical roller positioned within a roller seat of a dispenser body according to another embodiment of the present invention illustrating an inner gel container in dashed lines positioned within an outer gel container housing;

FIG. 11 is an exploded perspective view of a dispenser according to another embodiment of the present invention;

FIG. 12 is perspective view of a dispenser having a gel measuring member associated therewith according to yet another embodiment of the present invention;

FIG. 13 is a right side elevational view of a dispenser according to yet another embodiment of the present invention;

FIG. 14 is a left side elevational view of a dispenser according to yet another embodiment of the present invention;

FIG. 15 is a top plan view of a dispenser according to an embodiment of the present invention;

FIG. 16 is a bottom plan view of a dispenser according to an embodiment of the present invention;

FIG. 17 is a front elevational view of a dispenser according to an embodiment of the present invention;

FIG. 18 is a rear elevational view of a dispenser according to an embodiment of the present invention;

FIG. 19 is an enlarged view of a portion of an inner gel container housing, an outer gel housing, and a valve being connected to a dispenser body according to an embodiment of the present invention;

FIG. 20 is a sectional view of a dispenser taken along line 20-20 of FIG. 17 according to an embodiment of the present invention;

FIG. 21 is a sectional view of a dispenser without an inner gel container according to an embodiment of the present invention;

FIG. 22 is a front perspective view a dispenser still another embodiment of the present invention;

FIG. 23 is a rear perspective view of a dispenser having a clothing attachment member detached from the dispenser further according to an embodiment of the present invention;

FIG. 24 is a front perspective view of a dispenser according to a further embodiment of the present invention;

FIG. 25 is a rear perspective view of a dispenser according to a further embodiment of the present invention;

FIG. 26 is a rear perspective view of a dispenser having a clothing attachment member detached from a dispenser according to a further embodiment of the present invention;

FIG. 27 is an environmental view of healthcare personnel using a dispenser according to an embodiment of the present invention;

FIG. 27A is an enlarged side elevational view of healthcare personnel using the dispenser in FIG. 27 according an embodiment of the present invention;

FIG. 28 is an environmental view of healthcare personnel sanitizing hands according to an embodiment of the present invention;

FIG. 29 an environmental view of healthcare personnel using a dispenser to wipe gel residue on outer surfaces of a flexible fabric housing according to an embodiment of the present invention;

FIG. 29A is an enlarged side elevational view of a dispenser in FIG. 29 according to an embodiment of the present invention;

FIG. 30 is an sectional view along line 30-30 of FIG. 24 according to an embodiment of the present invention;

FIG. 31 is a perspective view of a kit of plurality of disposable wearable gel dispensers according to an embodiment of the present invention; and

FIG. 32 is an exploded perspective view of a kit of a wearable gel dispenser having a plurality of replaceable and refillable outer gel container housings and a dispenser body therein according to another embodiment of the present invention.

### DETAILED DESCRIPTION OF THE INVENTION

Embodiments of the present invention will now be described more fully hereinafter with reference to the accompanying drawings, which illustrate embodiments of the present invention. This invention may, however, be embodied in many different fowls and should not be construed as limited to the illustrated embodiments set forth herein; rather, these embodiments are provided so that this disclosure will be thorough and complete, and will fully convey the scope of the invention to those skilled in the art. Like numbers refer to like elements throughout.

Although referred to by specific example, various other individual or personnel, e.g., industry, office, manufacturing, government, can also use the dispensers as will be understood by those skilled in the art, and the healthcare personnel example and reference herein includes these other areas of use and other personnel in these environments.

Embodiments of the present invention relate to apparatus and methods for dispensing sanitizing gel to one or more hands 105 of healthcare personnel 101 when the dispenser is attached to clothing or clothing accessories of healthcare personnel 103. Embodiments of the present invention, for example, can aid in reducing the spread of bacteria and infections by dispensing sanitizing gel using a wearable gel dispenser 100, 200, 400, 500, 600. The wearable gel dispenser 100, 200, 400, 500, 600, according to embodiments of the present invention, is actuated by an intuitive and natural hand motion. Embodiments of the wearable gel dispenser can include, for example, an ellipsoidal roller 120, 220, 320 adapted to dispense the gel when actuated by the hand motion of healthcare personnel to supply gel 50 to the ellipsoidal roller 120, 220, 320 and allow the gel to be applied in a rolling motion to the hands 105 of healthcare personnel. Accordingly, embodiments of the present invention can provide, for example, cost-effective wearable sanitizing gel dispensers that are suitable for the healthcare environment, or any other environment that requires frequent hand sanitizing. For example, as will be understood by those skilled in the art, embodiments of the present invention can aid in effectively reducing the spread of bacteria in schools, professional kitchens, dentist offices, and hospitals.

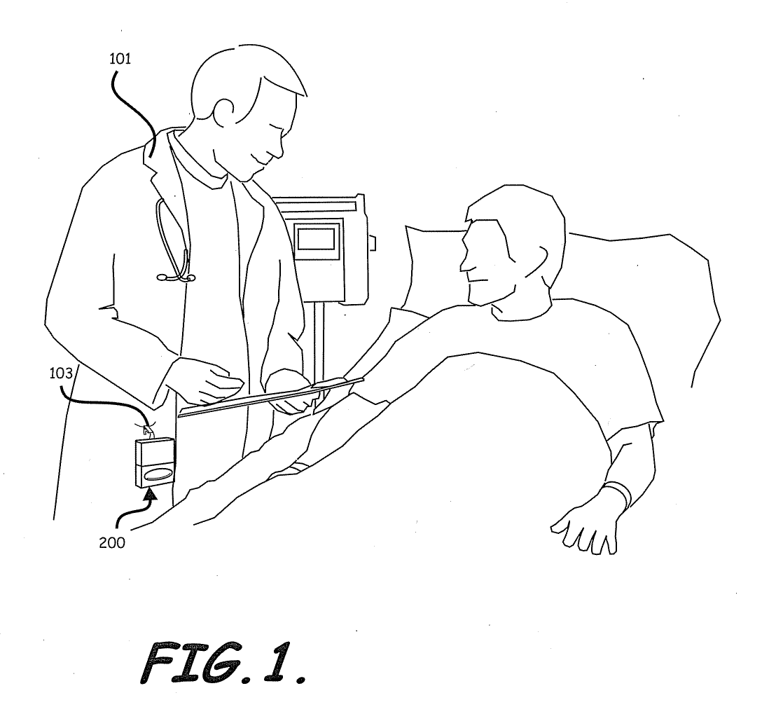

As illustrated in FIGS. 1-3 and FIGS. 27-28, for example, embodiments of the present invention can be worn by healthcare personnel 101 throughout their shift to sanitize their hands 105 and assist in meeting with the Center for Disease and Control (“CDC”) hand hygiene guidelines. The CDC hand hygiene guidelines recommend healthcare personnel sanitize their hands before and after every contact with a patient. See, e.g., Morbidity and Mortality Weekly Report: Recommendations and Reports Oct. 25, 2002, Vol. 51 No. RR-16. The World Health Organization (“WHO”) recommends that healthcare personnel sanitize their hands (1) before every patient contact; (2) before an aseptic task; (3) after risk exposure to bodily fluids (e.g., changing a patient's bed linen); (4) after patient contact; and (5) after contact with patient surroundings (e.g., bed, door knobs, etc.). See World Health Organization, “Five Moments for Hand Hygiene.”

If healthcare personnel of a hospital, for example, visits with fifteen to twenty (15-20) patients per eight hour shift in a hospital or clinic, to comply with CDC hand hygiene guidelines the average healthcare personnel should sanitize their hands on average thirty to eighty (30-80) times per shift. Accordingly, in order to comply with hand hygiene guidelines in the healthcare environment, the gel dispensing should be in an intuitive and natural manner to promote frequent sanitization. Although healthcare personnel are warned against wiping their hands against their scrubs, healthcare personnel subconsciously do it anyway. By dispensing gel using a substantially similar motion, embodiments of the wearable sanitizing gel dispenser 100, 200, 400, 500, 600 aids in enabling healthcare personnel 101 to comply with CDC and WHO hand hygiene guidelines in an unburdensome, intuitive and cost-effective manner.

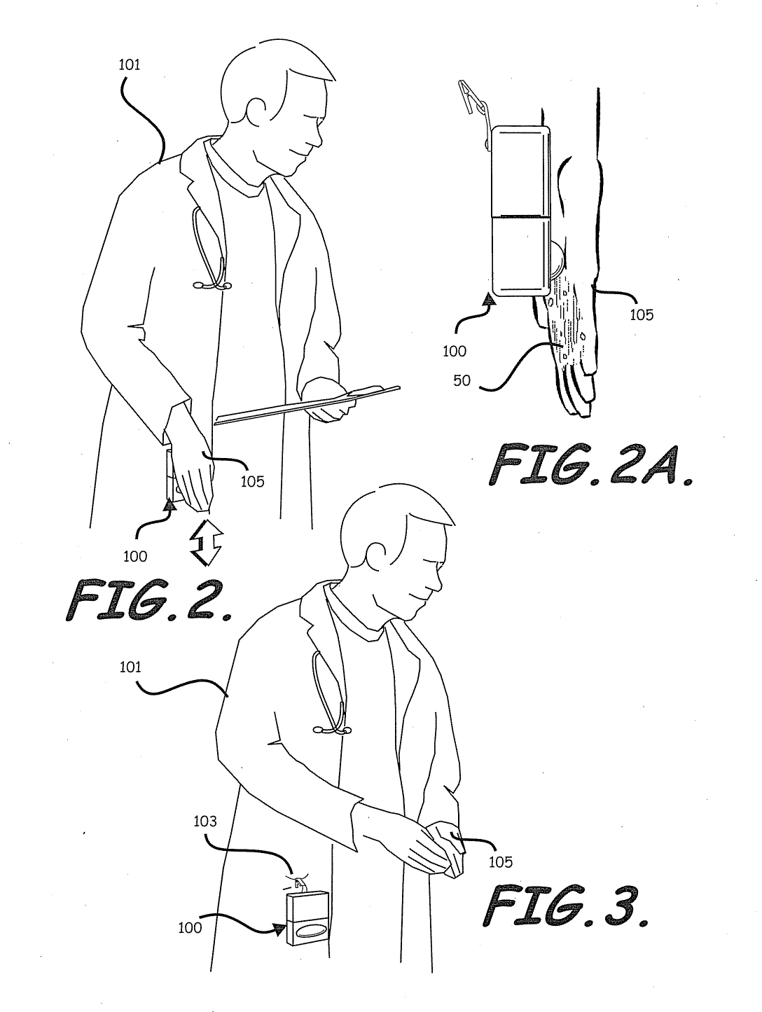

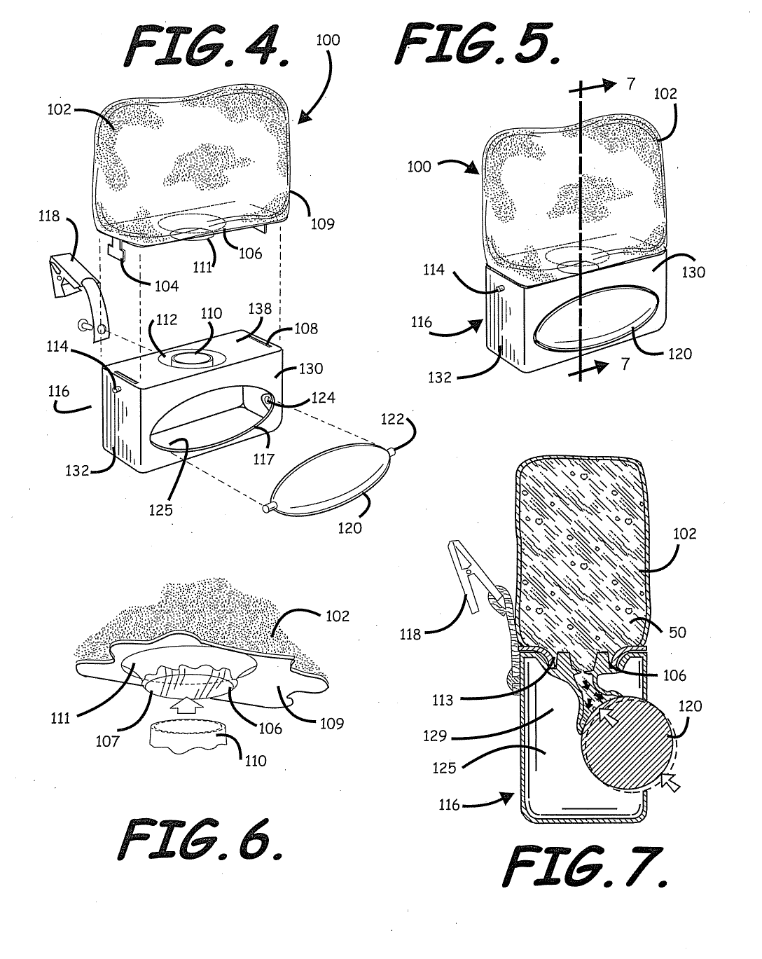

As illustrated in FIGS. 4-7, an embodiment of the present invention includes a sanitizing gel dispenser 100 to dispense sanitizing gel 50 to one or more hands 101 of healthcare personnel when the dispenser is attached to clothing or clothing accessories 103 of healthcare personnel. The embodiment includes an outer gel container housing 102 adapted to position and house gel 50. The embodiment also includes, for example, a dispenser body 116 adapted to house an ellipsoidal roller 120 that is actuated by a selective hand motion by healthcare personnel 101. As illustrated in FIGS. 2 and 2A, the selective hand motion is a natural up or down wiping motion that causes the ellipsoidal roller 120 to rotatingly dispense gel 50 to one or more hands 105 of healthcare personnel. Because the selective hand motion is an intuitive motion, embodiments of the present invention promotes the frequent use of sanitizing gel. As a result, embodiments of the present invention can assist in meeting hand hygiene compliance in the healthcare environment.

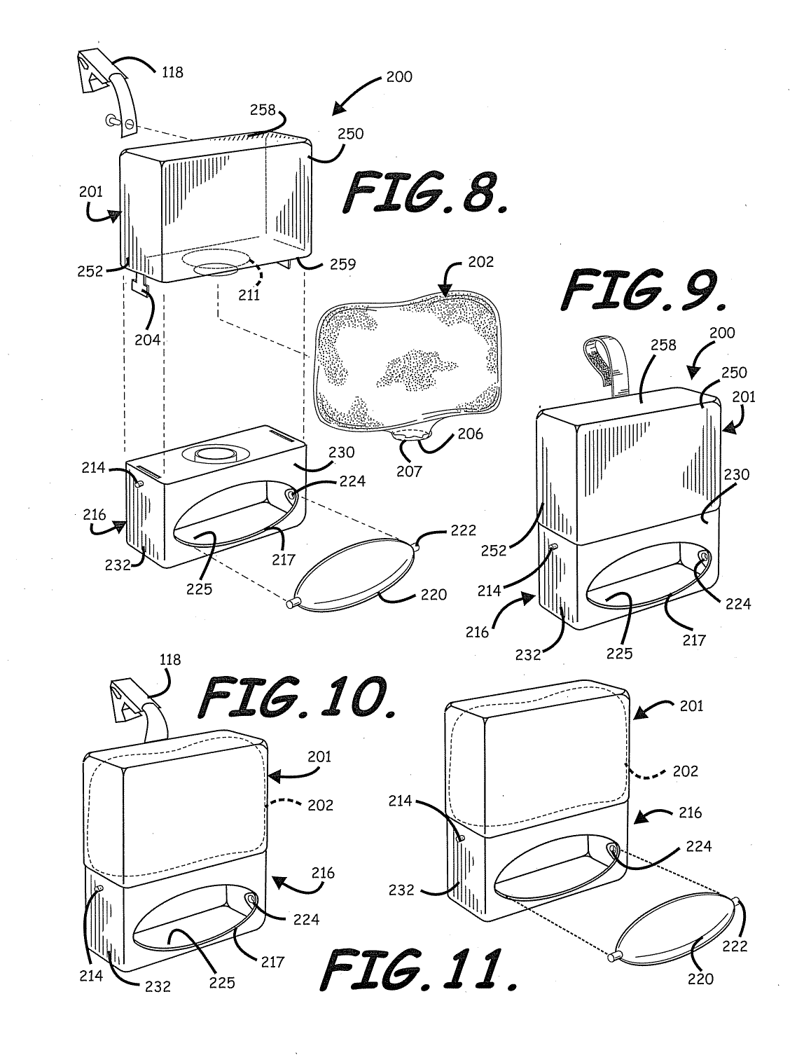

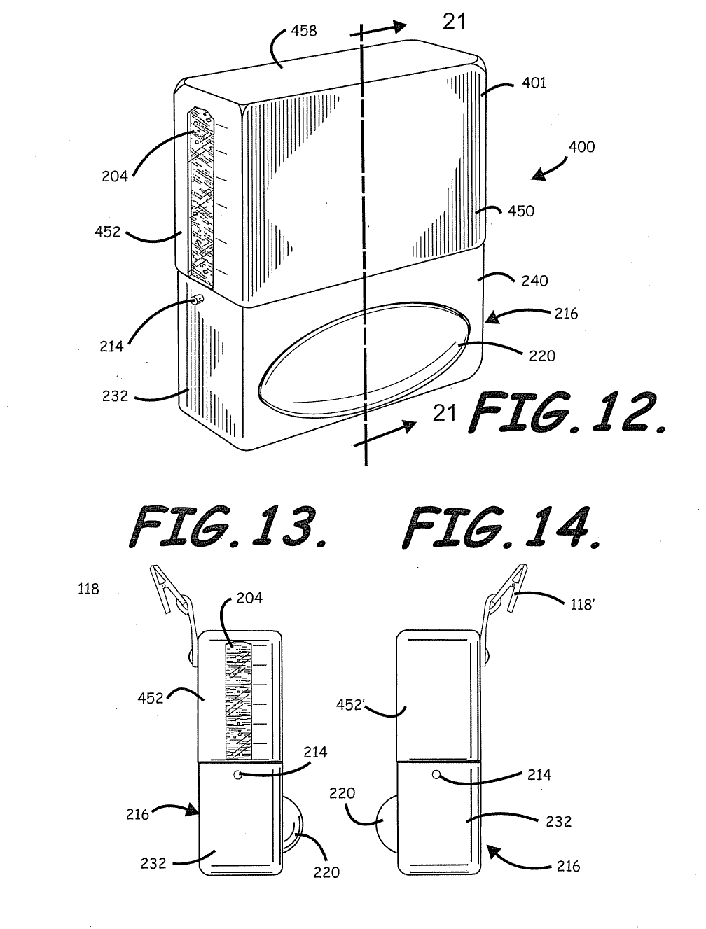

Another embodiment of the present invention, as illustrated in FIGS. 8-11, includes both an inner and outer housing. The outer gel container housing 201 is adapted to position and house an inner gel container 202. The embodiment (as also illustrated in FIGS. 2 and 2A) is actuated by a selective hand motion by healthcare personnel. This selective hand motion causes the ellipsoidal roller 220 to rotatingly dispense gel to one or more hands 105 of healthcare personnel. The outer gel container housing 201, according to the an embodiment of the present invention, can be, for example, made out of an inflexible plastic material. In the alternative to the inner gel container housing 202, according to embodiments of the present invention, the outer gel container housing 202 can be refilled with gel 50 through a chamber 211 disposed on a bottom wall 259 of the outer gel container housing 201 when the amount of gel has reached a undesirable amount.

The outer gel housing 201, according to embodiments of the present invention, includes a front wall 250, a back wall 254, and side walls 252. The outer gel housing 201 further includes a bottom wall 259 having a chamber 211 that protrudes outwardly. The chamber 211 can, for example, be adapted to position the aperture 206 of the inner gel container 202. The outer gel housing 201 further includes linking members 204 to detectably connect the outer gel container housing 201 to the dispenser body 216. As illustrated in FIG. 12, another embodiment of the outer gel housing 101 includes, for example, a measuring member 204 to measure the amount of gel remaining in the outer gel housing 401. The measuring member 204, for example, can be disposed on one or more of the side walls, 425′ to enable a user to visualize how much gel remains. Similar to other embodiments of the outer gel housing, this embodiment of the outer gel housing 401 include a front wall 450, a back wall 454, and side walls 452, 425′.

The outer gel container housing 102, according to an embodiment of the present invention, is adapted to position and house gel 50 therein. The outer gel container housing 102, according to the embodiment of the dispenser, has an aperture 106 formed in an end portion of outer gel container housing through which gel flows. The outer gel container housing 102, for example, can be a transparent flexible plastic that is rigid enough that it can hold its shape when positioned in the upright position such as polystyrene, or any other suitable plastic as will be understood by those skilled in the art. The outer gel container housing 102, according to embodiments of the present invention, has a substantially rectangular shape and the capacity of at least 100 milliliters of gel. This capacity should enable healthcare personnel to use the dispenser 100 to comply with the hand hygiene guidelines for at least half of an eight hour shift.

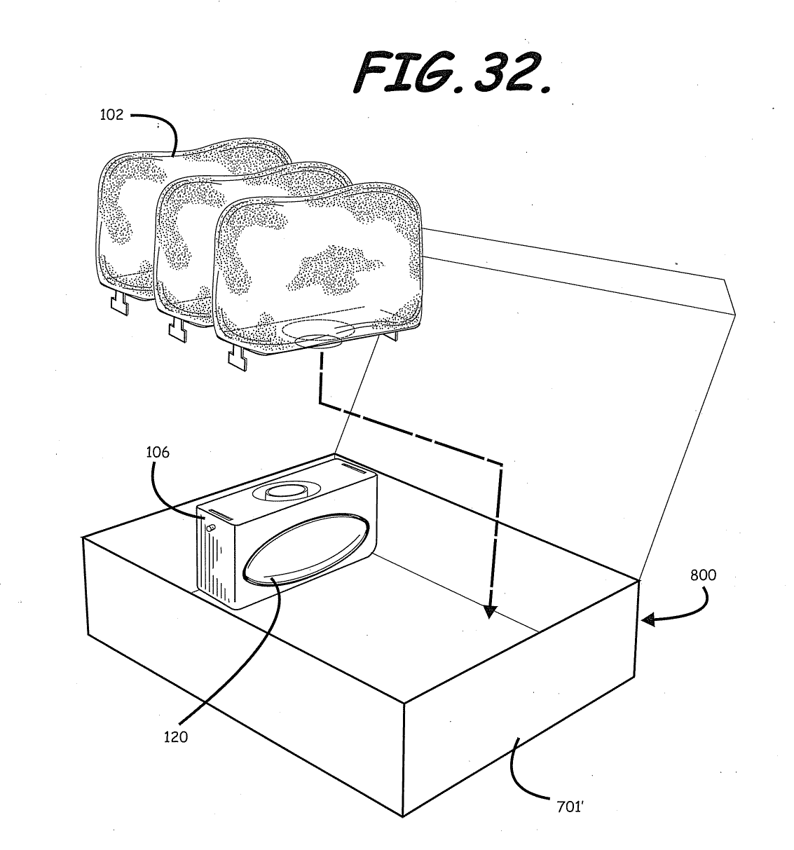

As illustrated in FIG. 32, a plurality of outer gel container housings 102 can be positioned within a container 701′ according to embodiments of a sanitizing dispenser kit 800. The sanitizing dispenser kit 700, 800 can also include in a dispenser body 116, 216 also positioned within the container 701′. The outer gel container housings 102 can be disposable cartridges of the dispenser or refillable. If the outer gel container housing 102 is refillable, gel is refilled through the aperture 106 of the outer gel container housing 102.

As illustrated in FIG. 8, according to an embodiment of the present invention, the inner gel housing 202 is positioned within the outer gel container housing 202. The inner gel housing 202 includes an aperture 206 that gel fluidly travels through. The aperture 206 is disposed at an end portion of the inner gel container 202. As illustrated in FIG. 19, when connected to the dispenser body 216, the aperture 206 of the inner gel housing 202 is disposed within the chamber 211 that protrudes outwardly from a bottom end portion of the outer gel container 202. The chamber 211, according to an embodiment of the present invention, matingly connects with a valve 210 to allow the gel to flowingly travel to the inner surfaces of the dispenser body 216.

As illustrated in FIG. 19, according to an embodiment of the present invention, the valve 210 punctures a penetrable material 207 extending across the aperture 206 when connected to the chamber 211. In the alternative to the inner gel container 202, according to embodiments of the invention, the outer gel housing 202 can be refillable such that gel 50 is refilled through the chamber 211 when the amount of the gel remaining in the outer gel container housing 202 reaches an undesirable amount. In this embodiment, the outer gel container housing 202 can be made of a inflexible, light-weight plastic material, as will be understood by those skilled in the art, such as, for example, polymer. In this embodiment, the penetrable material 207 can extend across the chamber 211, and punctured when it matingly connects with the valve 210.

As illustrated in FIG. 4, an embodiment of the present invention includes a dispenser frame 109 abutting an end portion of the outer gel container housing 102. The dispenser frame 109 is approximately the same length and width of the outer gel container housing 102, and is made out of a light-weight, ridged material to support the bottom end portion of the outer gel container housing 102. The dispenser frame 109 also includes a pair of linking members 104 positioned on opposite ends of a major axis, i.e., the longest axis, of the dispensing frame to enable the outer gel container housing 102 to detachable connect to a dispenser body 116. The linking members 104 are male connection members that matingly connect to a female linking mechanism 108.

As illustrated in FIG. 6, the dispenser frame 109, according to an embodiment of the invention, has a chamber 111 that is disposed between the linking members 104, and protrudes outwardly from a bottom end portion of the dispensing frame 109. The aperture 106 of the outer gel container housing 102 is positioned within the chamber 111 so that when the chamber 111 matingly connects with a valve 110, the valve 110 punctures a penetrable material 107 extending across the aperture 106 thereby allowing gel 50 to fluidly travel to the inner surfaces of the dispenser body 116.

According embodiments of the present invention, the dispenser body 116, 216, 316 connects to the outer gel container housing 102, 201, 401. The dispenser body 116, 216, 316 is a substantially rectangular shape, and is approximately the same length as the outer gel container housing 102, 202, 401. The dispenser body 116, 216, 316 can be made out of, for example, a light-weight polymeric material as will be understood by those skilled in the art. The dispenser body 116, 216, 316 is adapted to house an ellipsoidal roller 120, 220, 320 to rotatingly dispense gel to one or more hands 105 of healthcare personnel. As illustrated in FIG. 17, for example, the dispenser body 116, 216, 316 includes a front wall 130, 230, 330 having a front face with a substantially ellipsoidal cavity 117, 217, 317. The ellipsoidal roller 120, 220, 320 protrudes outwardly from the cavity when positioned within the dispenser body 116, 216, 316.

The dispenser body 116, 216, 316, according to embodiments of the present invention, also includes a roller seat 125, 225 formed in the front face of the front wall 130, 230, 230. The roller seat 125, 225, for example, can house the ellipsoidal roller 120, 220, 320 so that at least half of an outer circumference of medial portions of the ellipsoidal roller 120, 220, 320 is positioned within the inner surfaces of the dispenser body 116, 216, 316. As illustrated, for example, in FIGS. 13-14, the dispenser body 116, 216, 316 further includes side walls 132, 232, 332 connected to adjacent ends of the front walls 130, 230, 330. Attached to each side wall 132, 232, 332 includes a release mechanism 114, 214, 314 associated with the linking mechanism 118, 218, 318 to selectively detach the outer gel container housing 102, 202, 401 from the dispenser body 116, 216, 316. The release mechanism 114, 214, 314 can be, for example, in the form of a pair of buttons that, when pressed, cause the linking members 104, 204 to detach from the linking mechanism 108, 208.

As illustrated in FIG. 18, for example, embodiments of the dispenser body 116, 216, 316 also include a back wall 134, 234, 334 connected to the side walls 132, 232, 332. As illustrated in FIGS. 4 and 8, embodiments of the dispenser body 116, 216 include a top wall 138, 238 connected to upper ends of the side walls 132, 232, the back wall 134, 234 and the front wall 130, 230. The top wall 138, 238 includes the linking mechanism 108, 208 positioned on opposite ends along a major axis of the top wall of the dispenser body 116, 216. As discussed above, the linking mechanism 108, 208 enables the dispenser body 116, 216 to detachably connect to the outer gel container housing 102, 201, 401. The top wall of the dispenser body also includes an aperture seat 112, 212 to prevent gel from leaking between the aperture 106, 206 and the valve 110, 210. The aperture seat 112, 212, according to embodiments of the present invention, is disposed between the linking mechanism 108, 208, and substantially surrounds the valve 110, 210. When the valve 110, 210 matingly connects with the aperture 106, 206 the aperture seat 112, 212 substantially surrounds the chambers as well.

According an embodiments of the present invention, the dispenser body 116, 216 includes a bottom wall 139, 239 connected to bottom ends of the side walls 132, 232, the back wall 134, 234, and the front wall 130, 230. As illustrated in FIGS. 4, 8 and 11, embodiments of the present invention include one or more roller mounts such as a plurality of pin slots 124, 224 positioned within inner surfaces of the dispenser body matingly connected to a plurality of pins 122, 222 attached to opposite ends of the ellipsoidal roller 120, 220. The one or more roller mounts, as will be understood by those skilled in the art, can be of any suitable form including, for example, a roller frame positioned on the front wall 130, 230, 330 of the dispenser body to secure the roller 120, 220, 320 within the dispenser. The one or more roller mounts, as will be understood by those skilled in the art, can alternatively include one or more arms positioned within inner surfaces of the dispenser body matingly connected to depressions of the roller. The plurality of pin slots 124, 224, according to embodiments of the invention, are positioned adjacent opposite ends of a major axis of the cavity 117, 217 so that the ellipsoidal roller 120, 220 rotates about the major axis when secured within the dispenser body and actuated by the selective hand motion by healthcare personnel 101.

The valve 110, 210, according to embodiments of the present invention, is a circular shaped self-closing valve with an inside diameter in the range, for example, of approximately 0.50 to 0.75 inches. To dispense the gel, the valve matingly connects with the aperture of the outer gel container housing 102, or the inner gel container 202. The outer gel container housing 102 and the inner gel container 202 includes a penetrable material 107, 207 extending across the aperture 106, 206 to secure the gel within the inner surfaces of the outer gel container housing 102 and the inner gel container 202. As illustrated in FIGS. 6 and 19, when the outer gel container housing 102, or the inner gel container 202 is connected to the dispenser body 116, 216, the valve 110, 210 punctures the penetrable material 107, 207 to allow gel 50 to exit from the inner surfaces of the outer gel container 102 or the inner surfaces gel container 202 to the inner surfaces of the dispensing body 116, 216.

The ellipsoidal roller 120, 220, according to embodiments of the present invention, is adapted to connect to and seat within the roller seat 125, 225 of the dispenser body. To allow the roller 120, 220 to rotate about its major axis, i.e. its longest axis, the roller has a pair of pins 122 that are positioned on opposite ends of the roller along its major axis. The pins 122, 222 matingly connect to the pin slots 124, 224 and rotate therein when actuated by a selective hand motion.

The ellipsoidal shape of the roller 120, 220, 320 allows gel to rotatingly dispense to one or more hands 105 of healthcare personnel when actuated by a natural wiping motion in the up or down direction in orientation from the ground. The ellipsoidal roller 120, 220, 320 advantageously provides a larger surface area to efficiently dispense gel. The ellipsoidal shape of the roller also prevents the gel from seeping out the sides of the cavity 117, 217, 317 along the major axis. The positioning of the roller 120, 220, 320 within a cavity 117, 217, 317 on the front face 130, 230, 330 (instead of the bottom wall) also prevents gel from inadvertently seeping out. The gel 50 preferably contains 60%-95% alcohol, and dries within three seconds of being exposed to the atmosphere to further reduce the risk of inadvertent saturation. Commercial embodiments of the gel 50, for example, include Stat-Rinse Antiseptic Hand Gel.

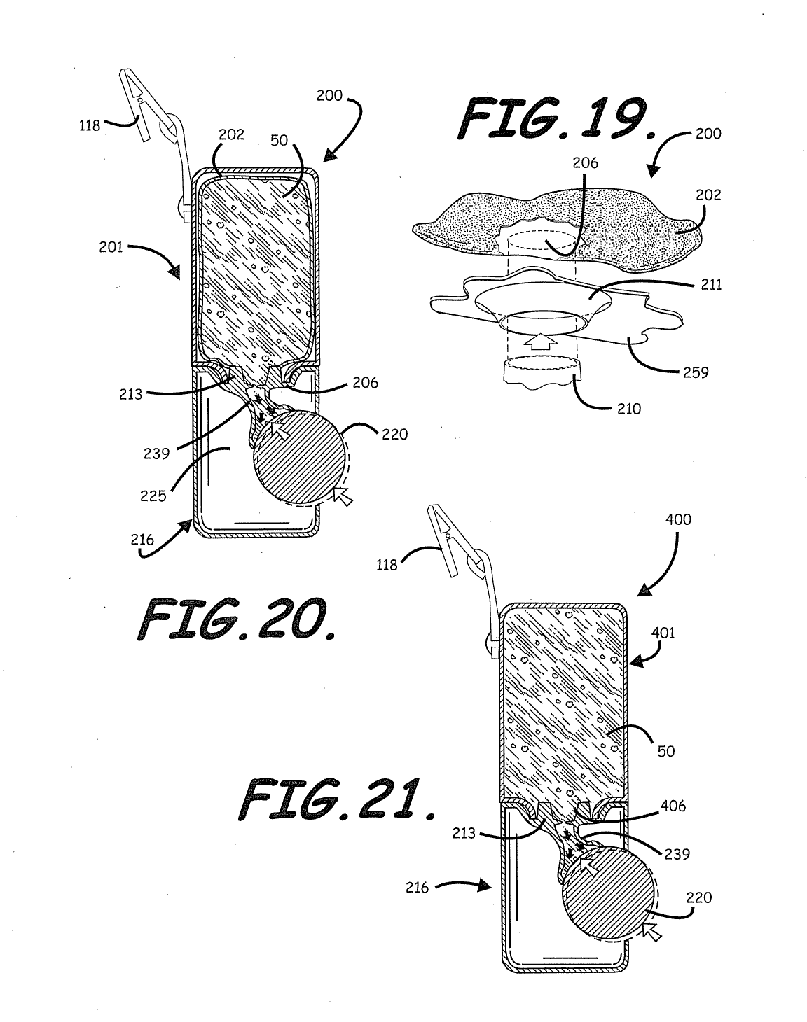

As illustrated in FIGS. 7, 20 and 21, the selective hand motion causes the ellipsoidal roller 120, 220 to move inward within the roller seat 125, 225. This causes the roller seat 125, 225 to press against the throat 129 and cause fluid communication with inner surfaces of the valve 113, 213. The inner surfaces of the valve 113, 213, according to embodiments of the present invention, extend downwardly within the inner surfaces of the dispenser body 116, 216 and touch the throat 129, 229 when the roller 120, 220 is actuated. When the roller 120, 220 is actuated, a selective portion of gel fluidly travels through the aperture 106, 206 through the valve 110, 210, through the throat 129, 229 and to the roller 120, 220 positioned within the roller seat 125, 225. When the roller 120, 220 is not actuated, the gel does not fluidly travel through the throat 129, 229.

This positioning and arrangement enables only allow a selective potion of gel to dispense and rollingly apply to one or more hands 105 of healthcare personnel 101. The selective portion of gel being significantly less than the amount of gel capable of being stored in the outer gel housing 102 or inner gel container 202. As illustrated, for example, in FIGS. 12 and 13, the dispenser 400, according to embodiments of invention, includes a measuring member 204 to measure the amount of gel remaining in the dispenser. The dispenser 100, 200, 400, 500, 600 is adapted to dispense up to fifty (50) individual dispenses of gel 50 to hands 105 of healthcare personnel.

To enable the dispenser to be wearable and portable, embodiments of the present invention include a clothing attachment member 118, 518 connected to the dispenser body 116, 216 to attach the gel dispenser to the clothing or clothing accessories 103 of healthcare personnel 101. In the alterative, the clothing attachment member 118, 518 can attach to the outer gel container. The clothing attachment member 118, 518 can be in any suitable forms, as will be understood by those skilled in the art, including but not limiting to a Layard, a clip to attach to clothing or clothing attachment, or fast and loop fastens such as Velcro®. The clothing attachment member 118, 518 should be so that the dispenser 100, 200, 400, 500, 600 is in an orientation on the clothing or clothing accessories 103 of healthcare personnel as to allow gravity to cause gel to flow towards the aperture 106, 206 and to the ellipsoidal roller 120, 220. Moreover, the size of the dispenser 100, 200, 400, 500, 600, according to embodiments of the invention, is preferably such that it readily can fit within the average size hand, and be worn without substantially interfering with the working activities of healthcare personnel.

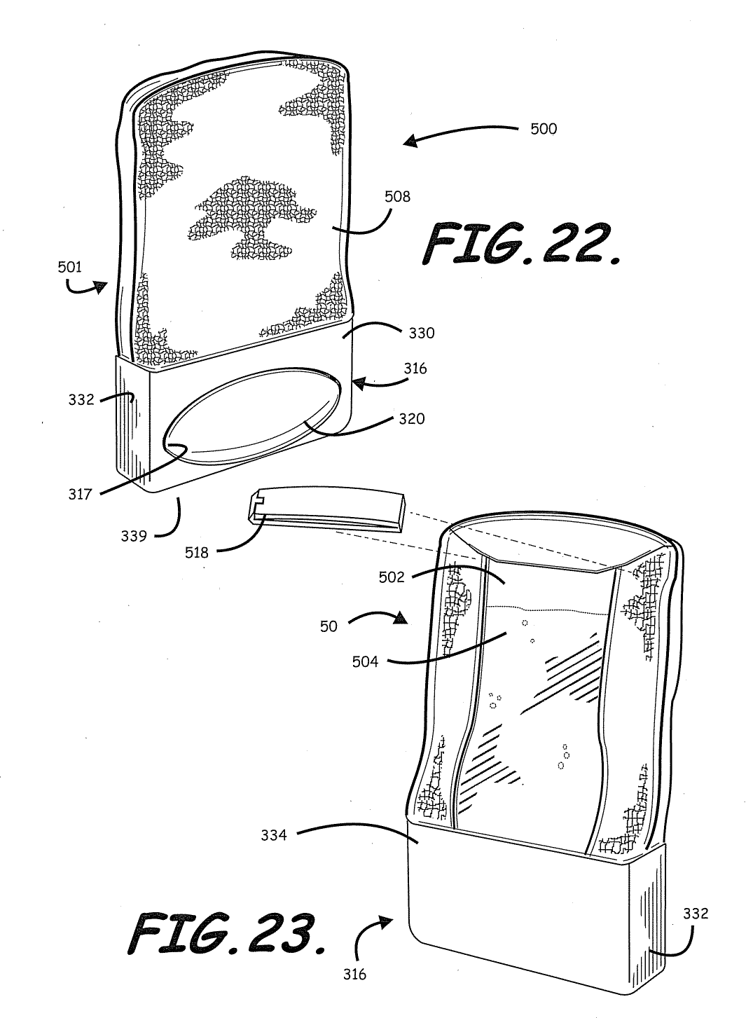

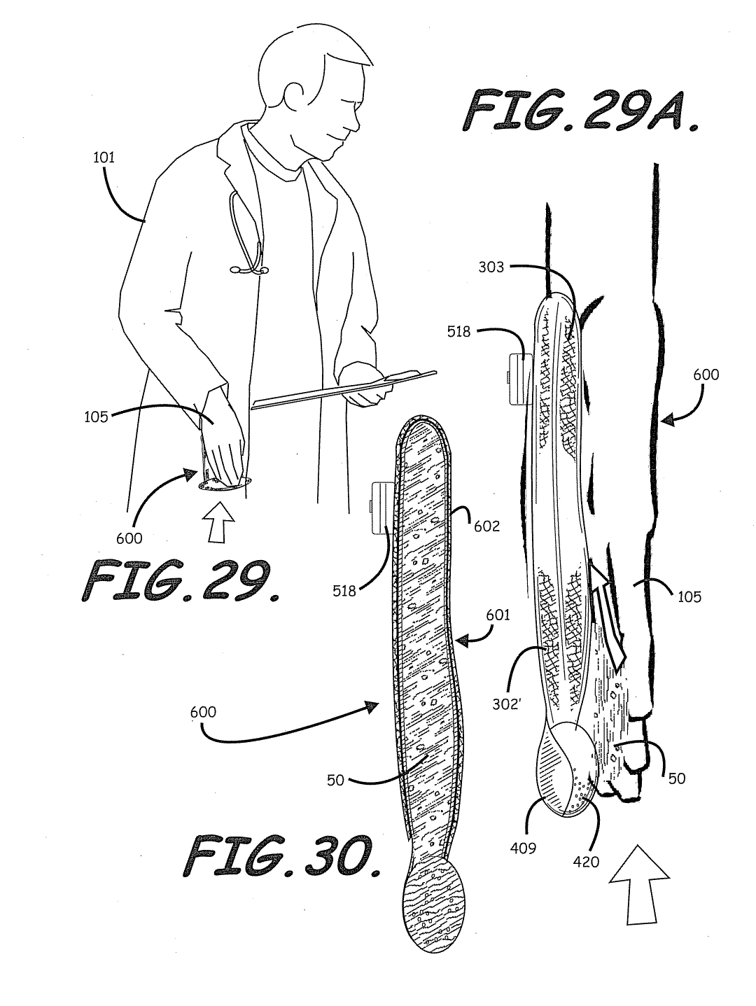

Another embodiment of the dispenser 500, as illustrated in FIG. 22, includes a flexible fabric housing 501 positioned to house an inner gel container 502 adapted to store sanitizing gel therein. As illustrated in FIGS. 29 and 29A, the flexible fabric housing 601 is adapted so that the outer surfaces of the fabric housing receives gel residue from hands 105 of healthcare personnel when hands of healthcare personnel are applied thereto. Embodiments of the flexible fabric housing can be made out of any suitable fabric, as will be understood by those skilled in the art, such as terry cloth, diaper cloth cotton, and other suitable cottons or wools. Preferably, at least the outer surfaces of the fabric housing is made out of an antibacterial fabric 303 to receive gel residue 50 from the hands of healthcare personnel 101, and to further sanitize said hands when applied thereto. The flexible fabric housing 601, according to embodiments of the present invention, does not have to completely enclose the inner gel container 302. For example, as illustrated in FIG. 23, the back face of the flexible fabric housing can be, for example, transparent to allow a user to see the amount of gel 50 remaining in the dispenser (a measuring member).

The antibacterial fabric 303, as understood by those skilled in the art, sanitizes surfaces it comes in contact with such as one or more hands 105 of healthcare personnel when applied thereto. Accordingly, an embodiment of the present invention can assist healthcare personnel 101 in sanitizing his or her hands 105 by wiping his or hands on the outer surfaces of the flexible fabric housing made out of the antibacterial fabric 303 alone when the dispenser 600 is attached to the clothing or clothing accessories 103 of healthcare personnel 101. The flexible fabric housing 303 made out of the antibacterial fabric can used to as a primary cleanser, e.g., used without the gel from the roller, or a secondary cleanser, e.g., used after gel is rollingly applied to the hands by the roller.

The dispenser 500, according to an embodiment of the present invention, can also include an ellipsoidal roller 320 in fluid communication with an aperture of the inner gel container 602. The ellipsoidal roller 320 is actuated by a selective hand motion of healthcare personnel to supply gel 50 to the roller 320, and allows the gel to rollingly apply to one or more hands 50 of healthcare personnel. See e.g., FIGS. 29 and 29A. The dispenser 300, according to the embodiment, also includes a clothing attachment member 518 to attach the dispenser 500 to the clothing or clothing accessories 103 of healthcare personnel in an orientation that as to allow gravity to cause the gel to flow towards the aperture of the inner gel container.

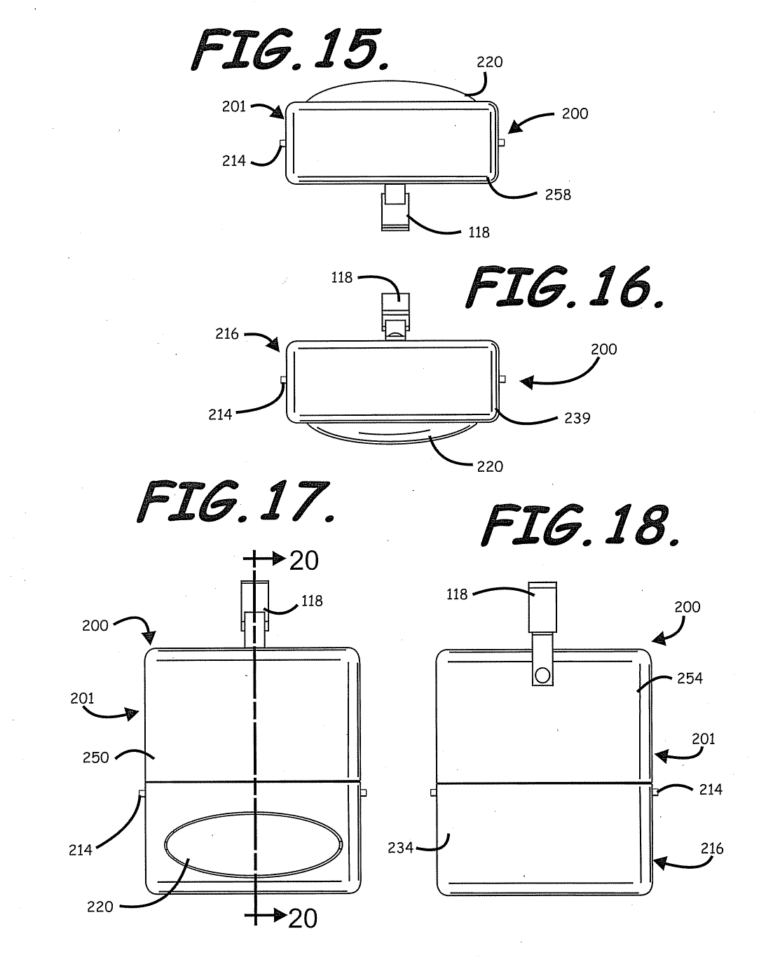

The dispenser body 316, according to an embodiment, is substantially rectangular, and is adapted to house the ellipsoidal roller 320 to rotating dispense gel to one or more hands 105 of healthcare personnel. Because the dispenser 500, according to this embodiment of the present invention, has a flexible fabric housing 501, the dispenser body 316 can be, for example, smaller than other embodiments of dispenser body disclosed herein. The dispenser body 316 includes a front wall 330 having a front face with a substantially ellipsoidal cavity 317 that the ellipsoidal roller 320 protrudes outwardly therefrom when positioned therein. The dispenser body 316, according to this embodiment of the present invention, also includes a roller seat formed in the front face 330 of the front wall. The roller seat, for example, can house the ellipsoidal roller 320 so that at least half of the ellipsoidal roller 320 is positioned within the inner surfaces of the dispenser body 316. See e.g., FIG. 15-16.

Similar to the other embodiments discussed herein, the dispenser body 316, according to this embodiment, also includes side walls 332, a back wall 334 and a bottom wall 339. The top wall 338 of the dispenser body, for example, can include a valve positioned thereon. The valve 210 matingly connects with the aperture 206 of the inner gel container 302 to allow the gel to flow therebetween. Substantially surrounding the valve 210, for example, is an aperture seat 212 to prevent the gel 50 from leaking between the aperture 206 and the valve 210.

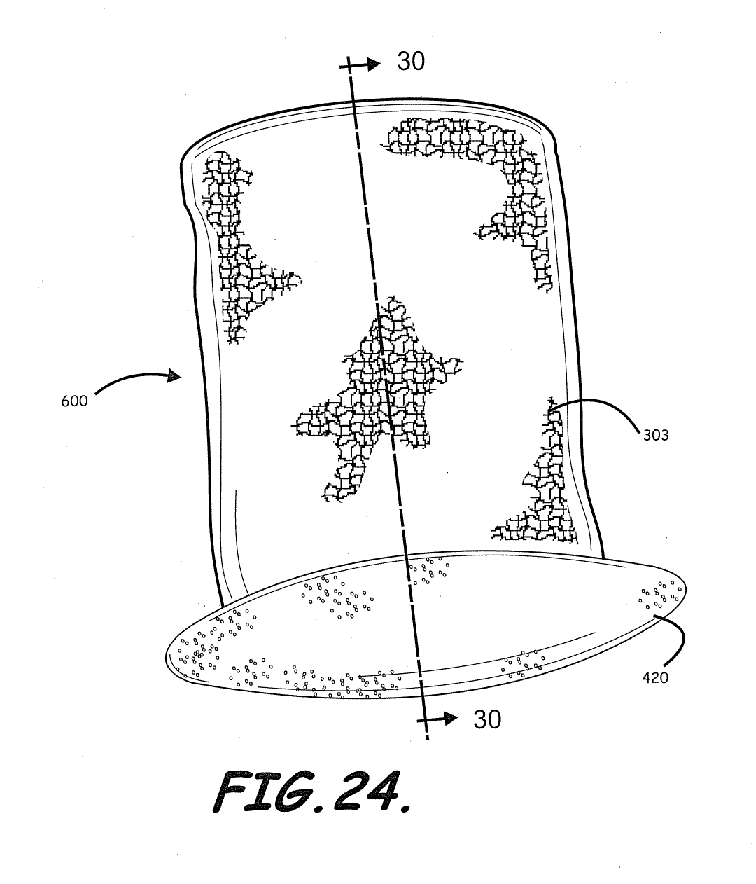

As illustrated in FIG. 24, another embodiment of the dispenser 600 also includes a flexible fabric housing 601 adapted to receive gel residue from hands 105 of healthcare personnel when said hands are applied thereto. Preferably at least the outer surfaces of the flexible fabric housing 601 are made out of an antibacterial material 303. The flexible fabric housing 301 also houses an inner gel container adapted to store sanitizing gel 50 therein. The inner gel container also includes an aperture 206 to allow the gel to exit from the inner gel container 602.

According to an embodiment of the present invention, the dispenser 600 also includes a dispensing sponge 420 in fluid communication with the aperture 106 of the inner gel container 602. The dispensing sponge 420, for example, can be elongated to provide a greater surface area for dispensing. As illustrated in FIG. 24, the elongated dispensing sponge 420 is positioned adjacent the flexible fabric housing 303. Embodiments of the invention can include, for example, a valve 110 positioned on a top end portion of the elongated sponge 420 adjacent the flexible fabric housing 601. The valve 110 matingly connects with the aperture 106 of the inner gel container 302 to allow fluid communication with the elongated sponge 420. In the alternative, the sponge 420 can be permanently affixed by a heat sink to the inner gel container 602. An aperture seat 112, for example, can prevent the gel 50 from leaking between the aperture 106 and the valve 110. The aperture seat 112 is disposed on the top end portion of the elongated sponge 420 and substantially surrounding the valve 110.

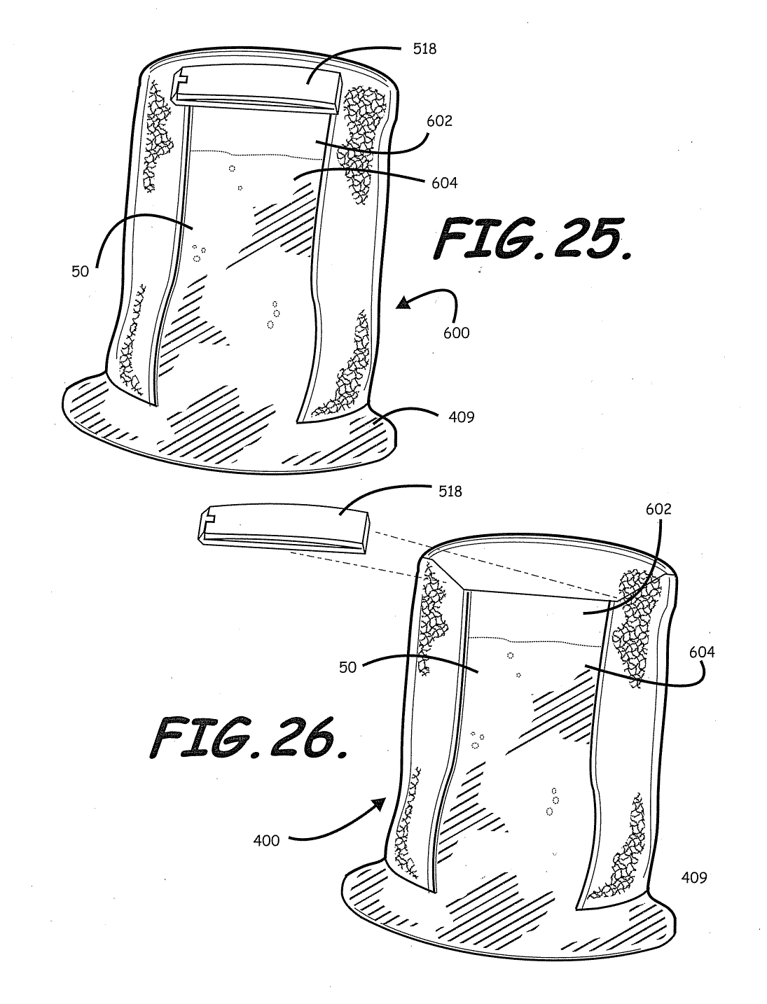

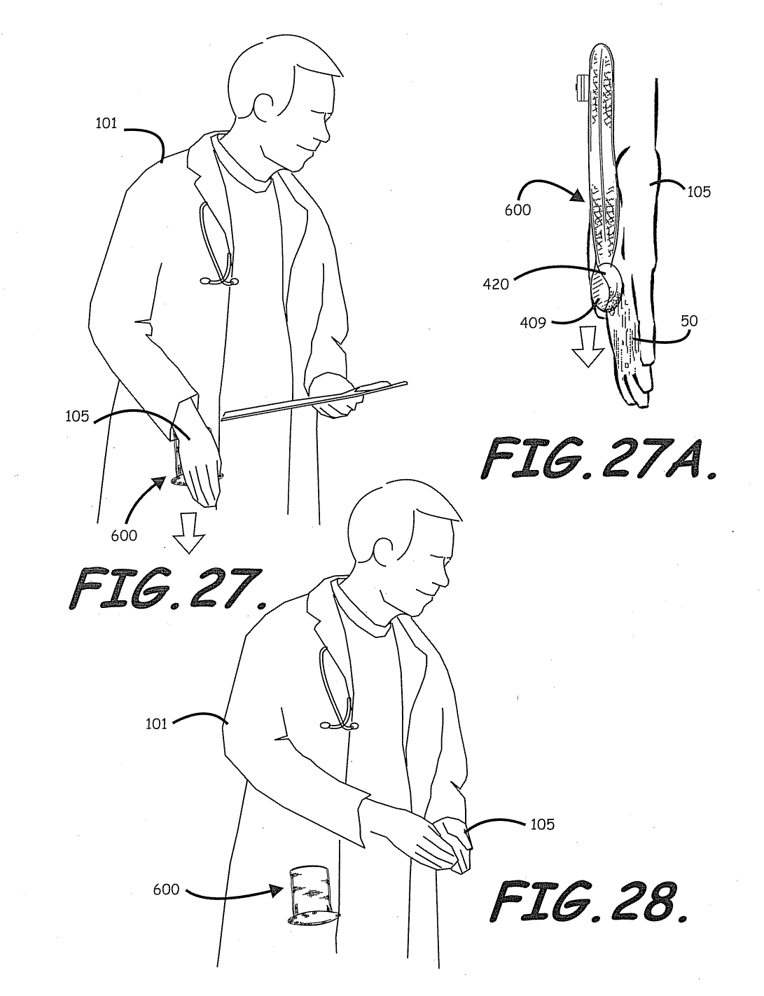

The dispenser 600, according to the embodiments of the present invention, is adapted to saturate the elongated sponge 420 with a selective amount of gel 50 to dispenser the gel to one or more hands 105 of healthcare personnel. The valve 110 is in fluid communication with the inner gel container 602 so that only a selective amount of gel enters within the inner surfaces of the elongated sponge 420 to thereby saturate it. As illustrated in FIGS. 25 and 26, the clothing attachment member 518, according to the embodiment of the present invention, is preferably attached to the back of the flexible housing 601 in an orientation on the clothing or clothing accessories 103 of healthcare personnel as to allow gravity to cause the gel to flow towards the aperture. This arrangement and positioning, for example, dispenses the selective amount of gel to the hands of healthcare personnel when healthcare personnel wipes said hands on the elongated sponge, or squeezes the elongated sponge. See e.g, FIGS. 27 and 27A. In the alternative, according to an embodiment of the present invention, the sponge 420 can be in constant communication with gel so that the sponge 420 is ready to dispense without squeezing or depressing the sponge 420. According to this embodiment, the sponge 420 sanitizes hands when wiped to the outer surfaces of the sponge. The saturation of the sponge 420, however, according the embodiment of the present invention, will not leak from the elongated housing 409.

As illustrated in FIGS. 25, 26 and 30 the dispenser 600, according to an embodiment, also includes an elongated housing 409 connected to and substantially surrounding the elongated sponge 420. The elongated housing 409 is adapted to house the elongated disposing sponge and prevent the gel from inadvertently saturating the clothing or clothing accessories of healthcare personnel. Moreover, preferred embodiments of the gel 50, as will be understood by those skilled in the art, dries within three seconds of exposure to the atmosphere to mitigate inadvertent saturation. This embodiment of the present invention can also include a measuring member 604 so that the back surface of the flexible fabric housing 601 is transparent to allow a user to see the amount of gel remaining in the dispenser.

Additionally, as illustrated in FIG. 29A, the hands of healthcare personnel can also be sanitized, according to the embodiment, by wiping them on the outer surfaces of the antibacterial material 303. The antibacterial fabric, as will be understood by those skilled in the art, on the outer surfaces of the flexible fabric housing 601 can be uses as a secondary cleaner to sanitize the hands—i.e., to receive gel residue from the hands and further sanitize, or as the primary cleaner to sanitize the hands.

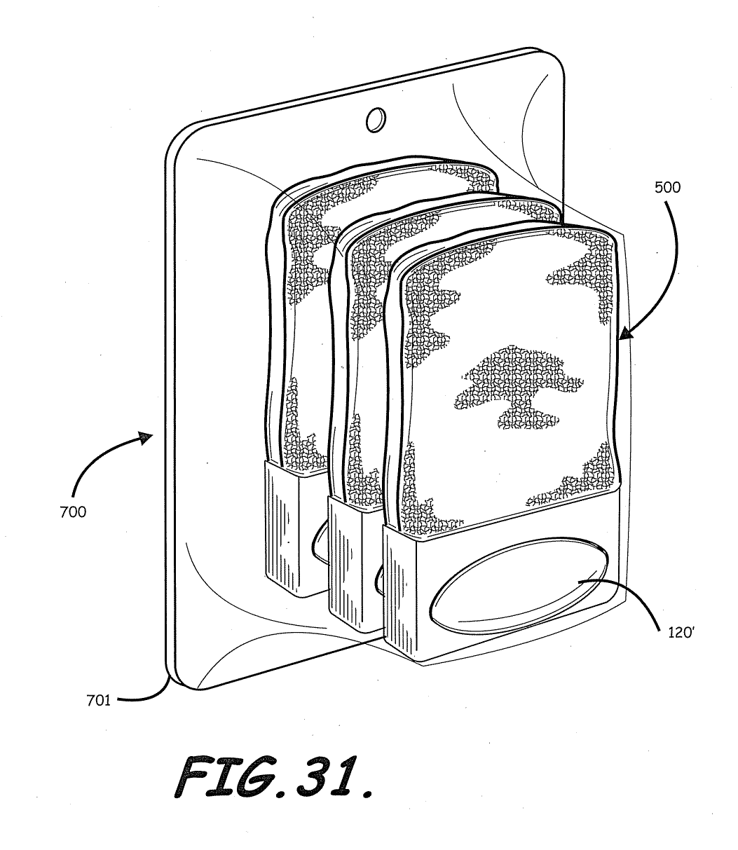

As illustrated in FIG. 31, embodiments of the present invention can include a dispenser kit 700 to dispense sanitizing gel 50 to one or more hands 105 of healthcare personnel when the dispenser 100, 200, 400, 500, 600 is attached to clothing or clothing accessories 103 of healthcare personnel. The kit 700 can include a container 701 containing a plurality of assembled dispensers 100, 200, 400, 500, 600 according to any of the embodiments of the present invention discussed herein. The plurality of dispensers 100, 200, 400, 500, 600, for example, can be disposable after a predetermined amount of individual dispenses, e.g., fifty individual dispenses, or after a predetermined amount of time such as after a shift of a healthcare personnel.

As illustrated in FIG. 32, embodiments of the present invention can include a dispenser kit 800 with a container 701′ containing a plurality of outer gel containers 102, 202, 401 adapted to position and house gel 50 therein. The container 701′ can also include a dispenser body 116, 216, 316 adapted to detachable connect to the outer gel containers 102, 201, 401. Both the plurality of outer gel containers 102, 201, 402 and the dispenser body 116, 216, 316 can be, for example, according to any of the embodiments of the present invention discussed herein. Accordingly, embodiments of the dispenser can be assembled by connected one of the plurality of outer gel containers 102, 201, 401 to the dispenser body 116, 216, 316. The plurality of outer gel 102, 201, 401 containers can be disposable, or refillable. The container 701, 701′ for example, as will be understood by those skilled in the art can be sealed to prevent outside containments from reaching one or more parts of the dispensers until the container 701, 107′ is unsealed.

Embodiments of the present invention can also include methods of dispensing sanitizing gel to one or more hands 105 of a healthcare personnel 101 when a sanitizing gel dispenser 100, 200, 400, 500, 600 is attached to the clothing or clothing accessories 103 of healthcare personnel 101. Embodiments of the method can include dispensing gel to hands healthcare personnel wearing a dispenser according to any of the embodiments discussed herein. For example, the method can include the step of, for example, providing an outer gel container housing 102, 201, 401 adapted to position and house an inner gel container 202,502, 602 therein. In the alternative, embodiments of a method of dispensing sanitizing gel 50 to one or more hands of healthcare personnel using a sanitizing gel dispenser 100, 200, 400, 500, 600 attached to the clothing or clothing accessories 103 of healthcare personnel 101 can providing an outer gel container 101, 202, 302, 402 housing adapted to position and house gel.

The method can also include the step of providing a dispenser body 116, 216, 316 with an ellipsoidal roller 120, 220, 320 positioned therein according to any of the embodiments of the apparatuses discussed herein. The method can further include the step of connecting the dispenser body 116, 216, 316 to the outer gel container housing 102, 201, 401. This connecting step can include using a valve 110, 210 disposed on a top end portion of the dispensing body 116, 216, 316 to puncture a penetrable material 107, 207 extended across of the aperture 106, 206 to allow gel to flow within the inner surfaces of the dispenser body.

Embodiments of the method can further include the step of attaching, the dispenser 100, 200, 400, 500 i.e., the connected dispenser body and the outer gel housing, to the clothing or clothing accessories 103 of healthcare personnel in an orientation that allow gravity to cause gel to flow towards the aperture 106, 206. Embodiments of the method can include the step of actuating the ellipsoidal roller 120, 220, 320 using a selective hand motion by healthcare personnel to supply gel to the ellipsoidal roller 120, 220, 320 with gel 50 and allow only a selective portion of gel to rollingly apply to one or more hands 105 of healthcare personnel 101. The selective portion of gel 50 being significantly less than the amount of gel capable of being stored in the outer gel housing container 102, 201, 401.

Embodiments of the method can include the step of apply one or more hands 105 of a healthcare personnel 101 to the outer surfaces of a flexible fabric 303 housing to sanitize said hands 105. As discussed above, this method of sanitizing can be used without the gel from the roller or elongated sponge, or after gel is rollingly applied to the hands by the roller.

Embodiments of the methods can include the step of providing a sponge 420 positioned adjacent a flexible fabric housing 303 and in fluid communication with an inner gel container 302 adapted to house gel 50. The method can further include sanitizing one or more hands 105 of healthcare personnel by squeezing the elongated sponge 420 or wiping the one or more hands to the outer surfaces of the elongated sponge 420.

This application claims benefit of, priority to, and is related to U.S. Provisional Patent Application No. 61/297346 filed on Jan. 22, 2010 titled, “Wearable Gel Dispensers,” which is incorporated herein by reference in its entirety.

Many modifications and other embodiments of the invention will come to the mind of one skilled in the art having the benefit of the teachings presented in the foregoing descriptions and the associated drawings. Therefore, it is to be understood that the invention is not to be limited to the illustrated embodiments disclosed, and that modifications and other embodiments are intended to be included within the scope of the appended claims.

That claimed is:

1. A sanitizing gel dispenser to dispense sanitizing gel when positioned therein to hands of healthcare personnel when the dispenser is attached to clothing or clothing accessories of healthcare personnel, the dispenser comprising:
an outer gel container housing adapted to position and house gel therein, the outer gel container housing having an aperture formed in an end portion thereof;
a dispensing frame abutting the end portion of the outer gel container housing and having linking members connected to opposite ends of a major axis of the dispensing frames, the dispensing frames having a chamber positioned between the linking members to position the aperture of the outer gel container housing therein;
the dispenser body connected to the outer gel container housing, the dispenser body including:
linking mechanism associated with the linking members to allow the dispenser body to detachable connect to the outer gel container housing, and
a roller seat formed in a front face of the dispenser body and adapted to secure an ellipsoidal roller within the dispenser body;
a release mechanism connected to the dispenser body to selectively detach the outer gel container housing from the linking mechanism of the dispenser body when connected thereto;
a valve disposed on an end portion of the dispenser body and adjacent the outer gel container housing when matingly connected to the aperture, the valve allowing fluid communication with the aperture of the outer gel container housing so that the dispensing body is adapted to receive gel flowing from the outer gel container housing;
an ellipsoidal roller connected to and seated in the roller seat of the dispenser body and shaped to allow gel to be readily dispensed to one or more hands of healthcare personnel by selective hand motion therefrom, the ellipsoidal roller adapted to dispense gel when actuated by the selective hand motion by healthcare personnel to supply gel to the ellipsoidal roller with gel and allow only a selective portion of gel to rollingly apply to one or more hands of healthcare personnel, the selective portion of gel being significantly less than the amount of gel capable of being stored in the outer gel housing container; and
a clothing attachment member connected to the dispenser body to attach the gel dispenser in an orientation on the clothing or clothing accessories of healthcare personnel so as to allow gravity to cause gel to flow toward the aperture of the outer gel container housing, into the dispenser body, and to the ellipsoidal roller.

2. A dispenser as defined in claim 1, wherein the dispenser body is substantially rectangular and further includes:
a front face wall with a substantially ellipsoidal cavity that the ellipsoidal roller protrudes outwardly therefrom when the ellipsoidal roller is positioned within the dispenser body; and
a plurality of pin slots positioned within inner surfaces of the dispenser body adjacent the cavity to secure the ellipsoidal roller within the dispenser body, and
wherein the ellipsoidal roller includes a plurality of pins matingly mounted to the plurality of pin slots, the plurality of pin slots being positioned on opposite ends of a major axis of the ellipsoidal roller so that the pins allow the ellipsoidal roller to rotate about the major axis when secured within the dispenser body and actuated by the selective hand motion by healthcare personnel.

3. A dispenser as defined in claim 2, wherein the ellipsoidal roller is seated in the roller seat of the dispenser body so that at least half of the outer circumference of medial portions of the ellipsoidal roller is positioned within inner surfaces of the dispenser body.

4. A dispenser as defined in claim 1, wherein the outer gel container housing further comprises:
a penetrable material extending across the aperture to secure the gel within inner surfaces of the outer gel container housing,
wherein the dispenser body further includes an aperture seat disposed on the end portion of the dispenser body substantially surrounding the valve to prevent gel from leaking between the aperture of the outer gel container housing and the valve when connected thereto, the aperture seat in communication with the chamber, and
wherein the valve extends outwardly from the end portion of the dispenser body and is adapted to puncture the penetrable material when in fluid communication with the aperture to allow the gel to exit from the inner surfaces of the inner gel container to inner surfaces of the dispensing body.

5. A dispenser as defined in claim 1, further comprising a measuring member connected to the dispenser body to measure the amount of gel remaining in the dispenser, and wherein the dispenser is adapted to dispense up to 50 individual dispenses of gel to hands of healthcare personnel.

6. A sanitizing gel dispenser to dispense sanitizing gel when positioned therein to hands of healthcare personnel when the dispenser is attached to clothing or clothing accessories of healthcare personnel, the dispenser comprising:
an inner gel container adapted to store sanitizing gel therein, the inner gel container including an aperture to allow the gel to exit from the inner gel container;
an outer gel container housing adapted to position and house the inner gel container therein, the outer gel container including a chamber to support the positioning of the aperture of the inner gel container therein;
linking members connected to opposite ends of an end portion of the outer gel container housing;
a dispenser body connected to the outer gel container housing, the dispenser body including:
linking mechanism associated with the linking members to allow the dispenser body to detachable connect to the outer gel container housing, and
a roller seat formed a front face of the dispenser body and adapted to secure an ellipsoidal roller within the dispenser body;
a release mechanism connected to the dispenser body to selectively detach the detachable outer gel container housing from the linking mechanism of the dispenser body when connected thereto;
a valve disposed on an end portion of the dispenser body and adjacent the outer gel container housing when matingly connected to the aperture, the valve allowing fluid communication with the aperture of the outer gel container housing so that the dispensing body is adapted to receive gel flowing from the outer gel container housing;
an ellipsoidal roller connected to and seated in the roller seat of the dispenser body and shaped to allow gel to be readily dispensed to one or more hands of healthcare personnel by selective hand motion therefrom, the ellipsoidal roller adapted to dispense gel when actuated by the selective hand motion by healthcare personnel to supply gel to the ellipsoidal roller with gel and allow only a selective portion of gel to rollingly apply to one or more hands of healthcare personnel, the selective portion of gel being significantly less than the amount of gel capable of being stored in the outer gel housing container; and
a clothing attachment member connected to the dispenser body to attach the gel dispenser in an orientation on the clothing or clothing accessories of healthcare personnel so as to allow gravity to cause gel to flow toward the aperture of the outer gel container housing, into the dispenser body, and to the ellipsoidal roller.

7. A dispenser as defined in claim 6, wherein the dispenser body is substantially rectangular and further includes:
a front face wall with a substantially ellipsoidal cavity that the ellipsoidal roller protrudes outwardly therefrom when the ellipsoidal roller is positioned within the dispenser body;
a plurality of pin slots positioned within inner surfaces of the dispenser body adjacent the cavity to secure the ellipsoidal roller within the dispenser body, and
wherein the ellipsoidal roller includes a plurality of pins matingly connected to the plurality of pin slots, the plurality of pin slots being positioned on opposite ends of a major axis of the ellipsoidal roller so that the plurality of pins allow the ellipsoidal roller to rotate about the major axis when secured within the dispenser body and actuated by selective hand motion from healthcare personnel.

8. A dispenser as defined in claim 7, wherein the ellipsoidal roller is seated in the roller seat of the dispenser body so that at least half an outer circumference of medial portions of the ellipsoidal roller is positioned within inner surfaces of the dispenser body.

9. A dispenser as defined in claim 6, wherein the inner gel container further comprises:
a penetrable material extending across the aperture to secure the gel within inner surfaces of the inner gel container;
wherein the dispenser body includes an aperture seat disposed on an upper end portion of the dispenser body substantially surrounding the valve to prevent gel from leaking between the aperture of the outer gel container housing and the valve when connected thereto, the aperture seat in communication with the chamber, and
wherein the valve extends outwardly from the upper end portion of the dispenser body and is adapted to puncture the penetrable material when in fluid communication with the aperture to allow the gel to exit from the inner surfaces of the inner gel container to inner surfaces of the dispensing body.

10. A dispenser as defined in claim 6, wherein the outer gel container housing is substantially rectangular and further includes:
a front wall;
a side walls connected to opposite adjacent ends of the front wall;
a back wall connected to the side walls and positioned across from the front wall;
a top wall connected to upper ends of the side walls, back wall, and front wall, and
a bottom wall connected to bottom ends of the side walls, back wall, and front wall, the bottom wall including:
linking members positioned on opposite ends along a major axis to allow the outer gel container housing to detachable connect to the dispenser body; and
a chamber disposed between the linking member and protruding outwardly to allow the gel to flow from inner surfaces of the outer gel container housing to that inner surfaces of the dispenser body.

11. A dispenser as defined in claim 6, further comprising a measuring member connected to the dispenser body to measure the amount of gel remaining in the dispenser, and wherein the dispenser is adapted to dispense up to 50 individual dispenses of gel to hands of healthcare personnel.

12. A sanitizing gel dispenser to dispense sanitizing gel when positioned therein to hands of an individual when the dispenser is attached to clothing or clothing accessories of the individual, the dispenser comprising:
an outer gel container housing adapted to store sanitizing gel therein, the outer gel container including:
an aperture to allow the gel to exit from the outer gel container;
a chamber to support the positioning of the aperture, and
linking members connected to opposite ends of an end portion of the outer gel container housing;
a dispenser body connected to the outer gel container housing, the dispenser body including:
linking mechanism associated with the linking members to allow the dispenser body to detachable connect to the outer gel container housing, and
a roller seat formed a front face of the dispenser body and adapted to secure an ellipsoidal roller within the dispenser body;
a release mechanism connected to the dispenser body to selectively detach the detachable outer gel container housing from the linking mechanism of the dispenser body when connected thereto;
a valve disposed on an end portion of the dispenser body and adjacent the outer gel container housing when matingly connected to the aperture, the valve allowing fluid communication with the aperture of the outer gel container housing so that the dispensing body is adapted to receive gel flowing from the outer gel container housing;
an ellipsoidal roller connected to and seated in the roller seat of the dispenser body and shaped to allow gel to be readily dispensed to one or more hands of the individual by selective hand motion therefrom, the ellipsoidal roller adapted to dispense gel when actuated by the selective hand motion by the individual to supply gel to the ellipsoidal roller with gel and allow only a selective portion of gel to rollingly apply to one or more hands of the individual, the selective portion of gel being significantly less than the amount of gel capable of being stored in the outer gel housing container; and
a clothing attachment member connected to the dispenser body to attach the gel dispenser in an orientation on the clothing or clothing accessories of the individual so as to allow gravity to cause gel to flow toward the aperture of the outer gel container housing, into the dispenser body, and to the ellipsoidal roller.

13. A dispenser as defined in claim 12, wherein the dispenser body further includes a substantially ellipsoidal cavity from which the ellipsoidal roller protrudes outwardly therefrom when the ellipsoidal roller is positioned within the dispenser body and a plurality of pin slots positioned within inner surfaces of the dispenser body adjacent the cavity to secured the ellipsoidal roller within the dispenser body, and
wherein the ellipsoidal roller includes a plurality of pins matingly connected with the plurality of pin slots, the plurality of pin slots being positioned on opposite ends of a major axis of the ellipsoidal roller so that the pins allow the ellipsoidal roller to rotate about the major axis when secured within dispenser body and actuated by the selective hand motion by healthcare personnel.

14. A sanitizing gel dispenser to dispense sanitizing gel when positioned therein to hands of an individual when the dispenser is attached to clothing or clothing accessories of the individual, the dispenser comprising:
an inner gel container adapted to store sanitizing gel therein, the inner gel container including an aperture to allow gel to exit from the inner gel container;
a flexible fabric housing positioned to house the inner gel container and adapted so that the outer surfaces of the flexible fabric housing receive gel residue from hands of the individual when hands of healthcare personnel are applied thereto;
an ellipsoidal roller in fluid communication with the aperture and adapted to dispense the gel when actuated by a selective hand motion by the individual to supply gel to the ellipsoidal roller with the gel and allow the gel to rollingly apply the gel to hands of the individual; and
a clothing attachment member affixed to the flexible housing to attach the gel dispenser in an orientation on the clothing or clothing accessories of the individual as to allow gravity to cause the gel to flow towards the aperture.

15. A dispenser as defined in claim 14, wherein at least the outer surface of the flexible fabric housing is an antibacterial fabric adapted to receive gel residue from hands of the individual when hands of the individual are applied thereto.

16. A dispenser as defined in claim 15, further comprising:
a dispenser body to house the ellipsoidal roller, the dispenser body with a substantially ellipsoidal cavity that the ellipsoidal roller protrudes outwardly therefrom when the ellipsoidal roller is positioned within the dispenser body;
pin slots positioned within inner surfaces of the dispenser body adjacent the cavity to secured the ellipsoidal roller within the dispenser body; and
and wherein the ellipsoidal roller includes pins are matingly connected with the pin slots, the pin slots positioned on opposite ends of a major axis of the ellipsoidal roller so that the pins allow the ellipsoidal roller to rotate about the major axis when secured within dispenser body and actuated by the selective hand motion by the individual.

17. A dispenser as defined in claim 15, wherein the ellipsoidal roller is positioned so that at least half of the ellipsoidal roller is positioned within inner surfaces of the dispenser body.

18. A dispenser as defined in claim 15, wherein the dispenser body includes an aperture seat disposed on an upper end portion of the dispenser body substantially surrounding the aperture to prevent gel from leaking between the aperture and the dispenser body.

19. A dispenser as defined in claim 15, wherein back surfaces of the inner gel container includes a measuring member to measure the amount of gel remaining in the dispenser, and wherein the dispenser is adapted to dispense up to 50 individual dispenses of gel to hands of the individual.

20. A dispenser as defined in claim 15, wherein the clothing attachment member is a hook and loop fastener.

21. A dispenser as defined in claim 15, wherein the dispenser further includes:
a valve connected to an end portion of the dispenser body adjacent the flexible fabric housing, the valve allowing fluid communication with the aperture of the inner gel container so that the ellipsoidal roller is adapted to receive gel from the inner gel container; and
an aperture seat disposed on the end portion of the ellipsoidal roller substantially surrounding the valve to prevent the gel from leaking between the aperture of the inner gel container and the valve when connected thereto.

22. A sanitizing gel dispenser to dispense sanitizing gel when positioned therein to hands of an individual when the dispenser is attached to clothing or clothing accessories of healthcare personnel, the dispenser comprising:
an inner gel container adapted to store sanitizing gel therein, the inner gel container including an aperture to allow the gel to exit from the inner gel container;
a flexible fabric housing positioned to house the inner gel container and adapted so that the outer surfaces of the flexible fabric housing receive gel residue from hands of the individual when hands of the individual are applied thereto;
an elongated dispensing sponge in fluid communication with the aperture and adapted to saturate the elongated sponge with the gel to dispense the gel to hands of the individual, the elongated dispensing sponge positioned to extend adjacent an end portion of the flexible fabric housing;
an elongated housing connected to the elongated dispensing sponge to house the elongated dispensing sponge and prevent the gel from inadvertently saturating the clothing or clothing accessories of the individual; and
a clothing attachment member affixed to the flexible housing to attach the gel dispenser in an orientation on the clothing or clothing accessories of the individual as to allow gravity to cause the gel to flow towards the aperture.

23. A dispenser as defined in claim 22, wherein at least the outer surface of the flexible fabric housing is an antibacterial fabric adapted to receive gel residue from hands of the individual when hands of healthcare personnel are applied thereto.

24. A dispenser as defined in claim 22, wherein the dispenser body includes an aperture seat disposed on an upper end portion of the elongated sponge substantially surrounding the aperture to prevent gel from leaking between the aperture and the elongated sponge.

25. A dispenser as defined in claim 22, wherein the dispenser further includes:
a valve connected to an end portion of the elongated sponge adjacent the flexible fabric housing, the valve matingly connects with the aperture of the inner gel container so that the elongated sponge is adapted to receive gel from the inner gel container; and
an aperture seat disposed on the end portion of the elongated sponge substantially surrounding the valve to prevent the gel from leaking between the aperture of the inner gel container and the valve when connected thereto.

26. A dispenser as defined in claim 22, wherein the clothing attachment member is a hook and loop fastener.

27. A dispenser as defined in claim 22, wherein back surfaces of the inner gel container includes a measuring gauge to measure the amount of gel remaining in the dispenser, and wherein the dispenser is adapted to dispense up to 50 individual dispenses of gel to hands of the individual.

28. A dispenser body adapted to house an ellipsoidal roller so that sanitizing gel is dispensed to one or more hands of an individual by a selective hand motion therefrom when the dispenser body is connected to an outer gel container housing and attached to clothing or clothing accessories of the individual, the dispenser body comprising:
a front wall having a front face with a substantially ellipsoidal cavity that the ellipsoidal roller protrudes outwardly therefrom when the ellipsoidal roller is positioned within the dispenser body;
a roller seat formed in the front face of the front wall and adapted to house the ellipsoidal roller so that at least half of the ellipsoidal roller is positioned within inner surfaces of the dispenser body;
side walls connected to opposite adjacent ends of the front wall;
a back wall connected to the side walls and positioned across from the front wall;
a top wall connected to upper ends of the side walls, back wall, and front wall, the top wall including:
linking mechanism positioned on opposite ends along a major axis to allow the dispenser body to detachable connect to the outer gel container housing, and
an aperture seat disposed between the linking slots, the aperture seat substantially surrounding a valve when connected thereto to prevent gel from leaking between an aperture of the outer gel container housing and the valve when connected thereto;
a bottom wall connected to bottom ends of the side walls, back wall, and front wall; and
pin slots positioned within the inner surfaces of the dispenser body adjacent the cavity to matingly connect with the ellipsoidal roller when positioned within the dispenser body.

29. A outer gel container housing adapted to position and house sanitizing gel therein so that the gel is dispensed to one or more hands of an individual by a selective hand motion therefrom when a dispenser body is connected to the outer gel container housing and attached to clothing or clothing accessories of the individual, the outer gel container housing comprising:
a front wall;
a side walls connected to opposite adjacent ends of the front wall;
a back wall connected to the side walls and positioned across from the front wall;
a top wall connected to upper ends of the side walls, back wall, and front wall, and
a bottom wall connected to bottom ends of the side walls, back wall, and front wall, the bottom wall including:
linking members positioned on opposite ends along a major axis to allow the outer gel container housing to detachable connect to the dispenser body; and
a chamber disposed between the linking member and protruding outwardly to allow the gel to flow from inner surfaces of the outer gel container housing to that inner surfaces of the dispenser body.

30. A dispenser kit to dispensing sanitizing gel when positioned therein to hands of an individual when the dispenser is attached to clothing or clothing accessories of the individual, the dispenser kit comprising:
a container;
a set of a plurality of outer gel container housing positioned within the container adapted to position and house gel therein, the outer gel having an aperture formed in an end portion of the outer gel container housing;
a dispenser body positioned in the container and adapted to be detachably connected to the outer gel container housing, the dispenser body including:
a roller seat formed in a front face of the dispenser body and adapted to secure an ellipsoidal roller within the dispenser body;
a release mechanism connected to the dispenser body to selectively detach the detachable outer gel container housing from the linking mechanism of the dispenser body when connected thereto;
an ellipsoidal roller connected to and seated in the roller seat of the dispenser body and shaped to allow gel to be readily dispensed to one or more hands of the individual by selective hand motion therefrom, the ellipsoidal roller adapted to dispense gel when actuated by the selective hand motion by the individual to supply gel to the ellipsoidal roller with gel and allow only a selective portion of gel to rollingly apply to one or more hands of the individual, the selective portion of gel being significantly less than the amount of gel capable of being stored in the outer gel housing container; and
a clothing attachment member connected to the dispenser body to attach the gel dispenser in an orientation on the clothing Or clothing accessories of the individual so as to allow gravity to cause gel to flow toward the aperture of the outer gel container housing, into the dispenser body, and to the ellipsoidal roller.

31. A dispenser kit as defined in claim 28, wherein each of the plurality of outer gel container housings includes:
a penetrable material extending across the aperture to secure the gel within inner surfaces of the outer gel container housing;
a valve disposed on an end portion of the dispenser body and adjacent the outer gel container housing when matingly connected to the aperture;
wherein the dispenser body further includes an aperture seat disposed on an upper end portion of the dispenser body substantially surrounding the valve to prevent gel from leaking between the aperture of the outer gel container housing and the valve when connected thereto; and
wherein the valve extends outwardly from the upper end portion of the dispenser body and is adapted to puncture the penetrable material when in fluid communication with the aperture to allow the gel to exit from the inner surfaces of the inner gel container to inner surfaces of the dispensing body.

32. A dispenser kit as defined in claim 28, wherein the dispenser body includes:
a substantially ellipsoidal cavity that the ellipsoidal roller protrudes outwardly therefrom when the ellipsoidal roller is positioned within the dispenser body;
a plurality of pins slots positioned within inner surfaces of the dispenser body adjacent the cavity to secured the ellipsoidal roller within the dispenser body; and
wherein the ellipsoidal roller includes a plurality of pins matingly connected to the plurality of pin slots, the plurality of pins slots being positioned on opposite ends of a major axis of the ellipsoidal roller so that the pins allow the ellipsoidal roller to rotate about the major axis when secured within dispenser body and actuated by the selective hand motion by healthcare personnel.

33. A dispenser kit to dispensing sanitizing gel when positioned therein to hands of the individual when the dispenser is attached to clothing or clothing accessories of the individual, the dispenser kit comprising:
a container;
a set of a plurality of dispensers, each dispenser including:
a outer gel container housing positioned within the container adapted to position and house gel therein, the outer gel having an aperture formed in an end portion of the outer gel container housing;
a dispenser body connected to the outer gel container and adapted to dispense gel to one or more hands of the individual, the dispenser body including a roller seat formed in a front face of the dispenser body and adapted to secure an ellipsoidal roller within the dispenser body;
an ellipsoidal roller connected to and seated in the roller seat of the dispenser body and shaped to allow gel to be readily dispensed to one or more hands of the individual by selective hand motion therefrom, the ellipsoidal roller adapted to dispense gel when actuated by the selective hand motion by the individual to supply gel to the ellipsoidal roller with gel and allow only a selective portion of gel to rollingly apply, to one or more hands of the individual, the selective portion of gel being significantly less than the amount of gel capable of being stored in the outer gel housing container; and
a clothing attachment member connected to the dispenser body to attach the gel dispenser in an orientation on the clothing or clothing accessories of the individual so as to allow gravity to cause gel to flow toward the aperture of the outer gel container housing, into the dispenser body, and to the ellipsoidal roller.

34. A method of dispensing sanitizing gel using a sanitizing gel dispenser to hands of an individual, the method comprising:
attaching the sanitizing gel dispenser to the clothing or clothing accessories of the individual, the dispenser including:
an outer gel container housing adapted to position and house gel therein, the outer gel container housing having an aperture formed in an end portion thereof;
a dispensing frame abutting the end portion of the outer gel container housing and having linking members positioned on opposite ends of a major axis of the dispensing frames, the dispensing frames having a chamber positioned between the linking members to position the aperture of the outer gel container housing therein;
an dispenser body connected to the outer gel container housing, the dispenser body including:
a linking mechanism associated with the dispensing frame to allow the dispenser body to detachable connect to the outer gel container housing, and
a roller seat foamed in a front face of the dispensing frame and adapted to secure an ellipsoidal roller within the dispenser body;
a release mechanism connected to the dispenser body to selectively detach the outer gel container housing from the linking mechanism of the dispenser body when connected thereto;
a valve matingly connected to an end portion of the dispenser body and adjacent the outer gel container housing when connected thereto, the valve allowing fluid communication with the aperture of the outer gel container housing so that the dispensing body is adapted to receive gel flowing from the outer gel container housing;
an ellipsoidal roller connected to and seated in the roller seat of the dispenser body and shaped to allow gel to be readily dispensed to one or more hands of personnel by selective hand motion therefrom; and
a clothing attachment member connected to the dispenser body to attach the gel dispenser in an orientation on the clothing or clothing accessories of the individual so as to allow gravity to cause gel to flow toward the aperture of the outer gel container housing, into the dispenser body, and to the ellipsoidal roller;
actuating the ellipsoidal roller using a selective hand motion by personnel to supply gel to the ellipsoidal roller with gel and allowing only a selective portion of gel to rollingly apply to one or more hands of the individual, the selective portion of the gel being significantly less than the amount of gel capable of being stored in the outer gel container housing.

35. A method of dispensing sanitizing gel using a sanitizing gel dispenser to hands of an individual, the method comprising:
attaching the sanitizing gel dispenser to the clothing or clothing accessories of the individual, the dispenser including:
an outer gel container housing adapted to store sanitizing gel therein, the outer gel container including an aperture where gel exits through;
linking members connected to opposite ends of an end portion of the outer gel container housing;
a dispenser body connected to the outer gel container housing, the dispenser body including:
linking mechanism associated with the linking members to allow the dispenser body to detachable connect to the outer gel container housing, and
a roller seat formed a front face of the dispenser body and adapted to secure an ellipsoidal roller within the dispenser body;
a release mechanism connected to the dispenser body to selectively detach the detachable outer gel container housing from the linking mechanism of the dispenser body when connected thereto;
a valve connected to an end portion of the dispenser body and adjacent the outer gel container housing when matingly connected thereto, the valve allowing fluid communication with the aperture of the inner gel container so that the dispensing body is adapted to receive gel from the inner gel container;
an ellipsoidal roller connected to and seated in the roller seat of the dispenser body and shaped to allow gel to be readily dispensed to one or more hands of the individual by selective hand motion therefrom, the ellipsoidal roller adapted to dispense gel when actuated by the selective hand motion by the individual to supply gel to the ellipsoidal roller with gel and allow only a selective portion of gel to rollingly apply to one or more hands of the individual, the selective portion of gel being significantly less than the amount of gel capable of being stored in the outer gel housing container; and
a clothing attachment member connected to the dispenser body to attach the gel dispenser in an orientation on the clothing or clothing accessories of the individual so as to allow gravity to cause gel to flow toward the aperture of the outer gel container housing, into the dispenser body, and to the ellipsoidal roller, and
actuating the ellipsoidal roller using a selective hand motion by the individual to supply gel to the ellipsoidal roller with gel and allowing only a selective portion of gel to rollingly apply to one or more hands of the individual, the selective portion of the gel being significantly less than the amount of gel capable of being stored in the outer gel container housing.

36. A method of dispensing sanitizing gel using a sanitizing gel dispenser to hands of an individual, the method comprising:
connecting an outer gel container housing adapted to position and house gel therein to a dispenser body, the dispenser body adapted to house an ellipsoidal roller and includes:
a linking mechanism to allow the dispenser body to detachable connect to the outer gel container housing, and
a roller seat adapted to secure an ellipsoidal roller within the dispenser body;
attaching a clothing attachment member connected the dispenser body to the clothing or clothing accessories of healthcare personnel;
actuating the ellipsoidal roller using a selective hand motion by the individual to supply gel to the ellipsoidal roller with gel and allowing only a selective portion of gel to rollingly apply to one or more hands of the individual, the selective portion of the gel being significantly less than the amount of gel capable of being stored in the outer gel container housing.

37. A method of dispensing sanitizing gel defined in claim 36, wherein the outer gel container housing has an aperture formed in an end portion thereof and is associated with a dispensing frame in communication with the end portion of the outer gel container housing and having a chamber to position the aperture of the outer gel container housing therein, the dispensing frame having linking members positioned on opposite ends of a major axis of the dispensing frames.

38. A method of dispensing sanitizing gel defined in claim 36, wherein the dispenser body includes:
linking mechanism associated with the linking members to allow the dispenser body to detachable connect to the outer gel container housing, and
a roller seat formed in a front face of the dispenser body and adapted to secure the ellipsoidal roller within the dispenser body.

39. A method of dispensing sanitizing gel defined in claim 36, wherein the outer gel container housing further comprises a penetrable material extending across the aperture to secure the gel within inner surfaces of the inner gel container, wherein the dispenser body includes an aperture seat disposed on an upper end portion of the dispenser body substantially surrounding a valve to prevent gel from leaking between the aperture of the outer gel container housing and the valve when connected thereto, the valve extends outwardly from the upper end portion of the dispenser body, and wherein the connecting step punctures the penetrable material across the aperture with the valve to allow the gel to exit from the inner surfaces of the outer gel container housing to inner surfaces of the dispensing body.

40. A method of dispensing sanitizing gel using a sanitizing gel dispenser to hands of an individual, the method comprising:
attaching the sanitizing gel dispenser to the clothing or clothing accessories of the individual, the dispenser including:
an inner gel container housing adapted to store sanitizing gel therein, the outer gel container including an aperture where gel exits through;
a flexible fabric housing adapted store and position the inner gel container housing, at least the outer surfaces of the flexible fabric housing made out of antibacterial fabric;
a dispenser body connected to the outer gel container housing, the dispenser body including:
a roller seat formed a front face of the dispenser body and adapted to secure an ellipsoidal roller within the dispenser body;
an ellipsoidal roller connected to and seated in the roller seat of the dispenser body and shaped to allow gel to be readily dispensed to one or more hands of the individual by selective hand motion therefrom, the ellipsoidal roller adapted to dispense gel when actuated by the selective hand motion by healthcare personnel to supply gel to the ellipsoidal roller with gel and allow only a selective portion of gel to rollingly apply to one or more hands of the individual, the selective portion of gel being significantly less than the amount of gel capable of being stored in the outer gel housing container; and
a clothing attachment member connected to the dispenser body to attach the gel dispenser in an orientation on the clothing or clothing accessories of the individual so as to allow gravity to cause gel to flow toward the aperture of the outer gel container housing, into the dispenser body, and to the ellipsoidal roller,
actuating the ellipsoidal roller using a selective hand motion by the individual to supply gel to the ellipsoidal roller with gel and allowing only a selective portion of gel to rollingly apply to one or more hands of the individual, the selective portion of the gel being significantly less than the amount of gel capable of being stored in the outer gel container housing; and
applying the one or more hands to the antibacterial fabric to receive gel residue from the one or more hands and further sanitize the one or more hands.
